(19) United States
(12) Patent Application Publication
(10) Pub. No.: US 2017/0232185 A1
(43) Pub. Date: Aug. 17, 2017

(54) STERILIZATION OF FLUID PATHS IN INJECTION SYSTEM

(71) Applicant: ACIST Medical Systems, Inc., Eden Prairie, MN (US)

(72) Inventors: Robert F. Wilson, Roseville, MN (US); Edward R. Miller, Eden Prairie, MN (US); Kendall R. Waters, Livermore, CA (US)

(21) Appl. No.: 15/045,898

(22) Filed: Feb. 17, 2016

(51) Int. Cl. A61M 5/00; A61M 5/50; A61M 5/145

(52) U.S. Cl. CPC A61M 5/007; A61M 5/14546; A61M 5/001; A61M 5/5086; A61M 5/14228

(57) ABSTRACT

A contrast injector system includes one or more devices for reducing or eliminating risk of cross-patient contamination. In particular, the contrast injector system includes at least one of a sterilization device, vibration device, and illuminator device positioned on a component of the contrast injector system, where the sterilization device, vibration device, and/or illuminator device is in communication with a console of the contrast injector system. The sterilization device has an energy emitter positioned to emit energy to one or more components of the system. The vibration device is positioned on a component of the system so as to induce acoustic vibrations on a surface of such component. The illuminator device includes a light source positioned to illuminate a component of the system.

### TECHNICAL FIELD

This disclosure relates generally to injection systems and more particularly to reducing or eliminating potential contamination in injection systems.

### BACKGROUND

Many medical procedures, such as angiographies, involve injecting a contrast media directly into a patient. Angiography is a procedure used in the diagnosis and treatment of cardiovascular conditions including abnormalities or restrictions in blood vessels. During angiography, a radiographic image of the heart or vascular structure is obtained by injecting contrast media through a catheter into a vein or artery of the patient. The injected contrast media can pass to vascular structures in fluid communication with the vein or artery in which the injection is made. X-rays are passed through the region of the body in which the contrast media was injected. The X-rays are absorbed by the contrast media, causing a radiographic outline or image of the blood vessel containing the contrast media.

A contrast injection system can be utilized to inject the contrast media into the patient during such medical procedures. Some contrast injection systems include multi-use components that may be used in multiple procedures, and thus with multiple patients. In theory, any time a component is used multiple times across different patients, it may be possible that during a particular procedure such one or more multi-use components of a contrast injection system become exposed to a patient's bodily fluid. If this were to ever occur, the potential would exist for cross-patient contamination if a previously exposed multi-use component were utilized subsequently with another, different patient.

### SUMMARY

This disclosure relates generally to reducing or eliminating risk of cross-patient contamination in a contrast injector system. Various embodiments provide for sterilization, prevention of contaminant accumulation (e.g. formation), and/or detection of contamination in a fluid path or other component of a contrast injector system. Use of one or more disclosed embodiments may allow for safe utilization of multi-use components within a contrast injector system.

One embodiment includes a contrast injector system having a sterilization device in communication with a console. In such embodiment, the sterilization device has an energy emitter positioned to emit energy to a component of the contrast injector system. The sterilization device can serve to help maintain a sterile barrier within the contrast injector system by rendering harmless one or more contaminants or other matter passing through the component receiving the energy emitted by the sterilization device.

Another embodiment includes a contrast injector system having a vibration device in communication with a console. In such embodiment, the vibration device can be disposed on a surface of a component of the contrast injector system so as to induce vibrations on the surface of the component. The vibration device can serve to help prevent formation of one or more contaminants (e.g., biofilm) within the component.

A further embodiment includes a contrast injector system having an illuminator device in communication with a console. In such embodiment, the illuminator device has a light source positioned to illuminate the contents within a component in the system. The illuminator device can serve to provide an indication that a component includes one or more contaminants, and allow such component to be replaced before being used on a subsequent, different patient. The illuminator device can additionally or alternatively serve to provide an indication that a particle present within a component is of a size that should not be present in the particular application, and thus indicate potential contamination within that component.

The contrast injector system in various described embodiments can include, in addition to one or more of the sterilization, vibration, and/or illuminator devices, a manifold having first and second fluid inlets, a fluid outlet, and a valve configured to switch between allowing fluid communication from the first and second fluid inlets to the fluid outlet. A first fluid communication line connecting a first fluid supply container in fluid communication with the first fluid inlet and a second fluid communication line connecting a second fluid supply container in fluid communication with the second fluid inlet can further be included. In addition, a reservoir main body can be part of the contrast injector system embodiments, where the reservoir main body is positioned on the second fluid communication line and configured to receive fluid from the second fluid supply container and communicate this received fluid to the second fluid inlet of the manifold. The console of the contrast injector system embodiments can also be in communication with the reservoir main body to control an operational parameter of the reservoir main body.

A contrast injector system utilizing one or more of the sterilization, vibration, and/or illuminator devices can serve to sterilize, prevent, and/or detect contaminants and thus provide various related benefits, including the reduction or elimination of a potential risk of cross-patient contamination. This may be accomplished by maintaining a sterile barrier between one or more single-use components and one or more multi-use components, so as to prevent migration of harmful contaminants past the sterile barrier. For example, a contrast injector system that utilizes at least one of each of the three devices (sterilization, vibration, and illuminator) can facilitate eradication (e.g, via a sterilization device) and prevention (e.g., via a vibration device) of contaminants as well as facilitate detection (e.g., via an illuminator device) of a contaminated fluid path.

The details of one or more examples are set forth in the accompanying drawings and the description below. Other features, objects, and advantages will be apparent from the description and drawings, and from the claims.

### BRIEF DESCRIPTION OF DRAWINGS

FIG. 1A is a perspective view of an embodiment of a contrast injector system.

FIG. 1B is a perspective view of another embodiment of a contrast injector system.

FIG. 2A is a diagram illustrating an example of a first operation of the contrast injector system.

FIG. 2B is a diagram illustrating an example of a second operation of the contrast injector system.

FIG. 2C is a diagram illustrating an example of a third operation of the contrast injector system.

FIG. 3A is a side sectional view illustrating exemplary operation of an inlet valve system and manifold during a first operation.

FIG. 3B is a side sectional view illustrating exemplary operation of an inlet valve system and manifold during a second operation.

FIG. 3C is a side sectional view illustrating exemplary operation of an inlet valve system and manifold during a third operation.

FIG. 3D is a side sectional view illustrating exemplary operation of an inlet valve system and manifold during a fourth operation.

FIG. 4A is a side section view illustrating exemplary operation of an inlet valve system during a first operation of the contrast injector system.

FIG. 4B is a side section view illustrating exemplary operation of an inlet valve system during a second operation of the contrast injector system.

FIG. 4C is a side section view illustrating exemplary operation of an inlet valve system during a third operation of the contrast injector system.

FIG. 5A is a schematic diagram of an embodiment of a sterilization device.

FIGS. 5B and 5C are schematic diagrams illustrating portions of contrast injector system embodiments having differing locations of the sterilization device of FIG. 5A.

FIG. 6A is a schematic diagram of an embodiment of a vibration device.

FIGS. 6B and 6C are schematic diagrams illustrating embodiments having differing locations of the vibration device of FIG. 6A.

FIG. 7A is a schematic diagram of an embodiment of an illuminator device.

FIGS. 7B-7E are schematic diagrams illustrating embodiments having differing locations of the illuminator device of FIG. 7A.

Various examples have been described. These and other examples are within the scope of the following claims.

### DETAILED DESCRIPTION

Various exemplary embodiments are described herein with reference to the accompanying drawing figures in which like numbers describe like elements.

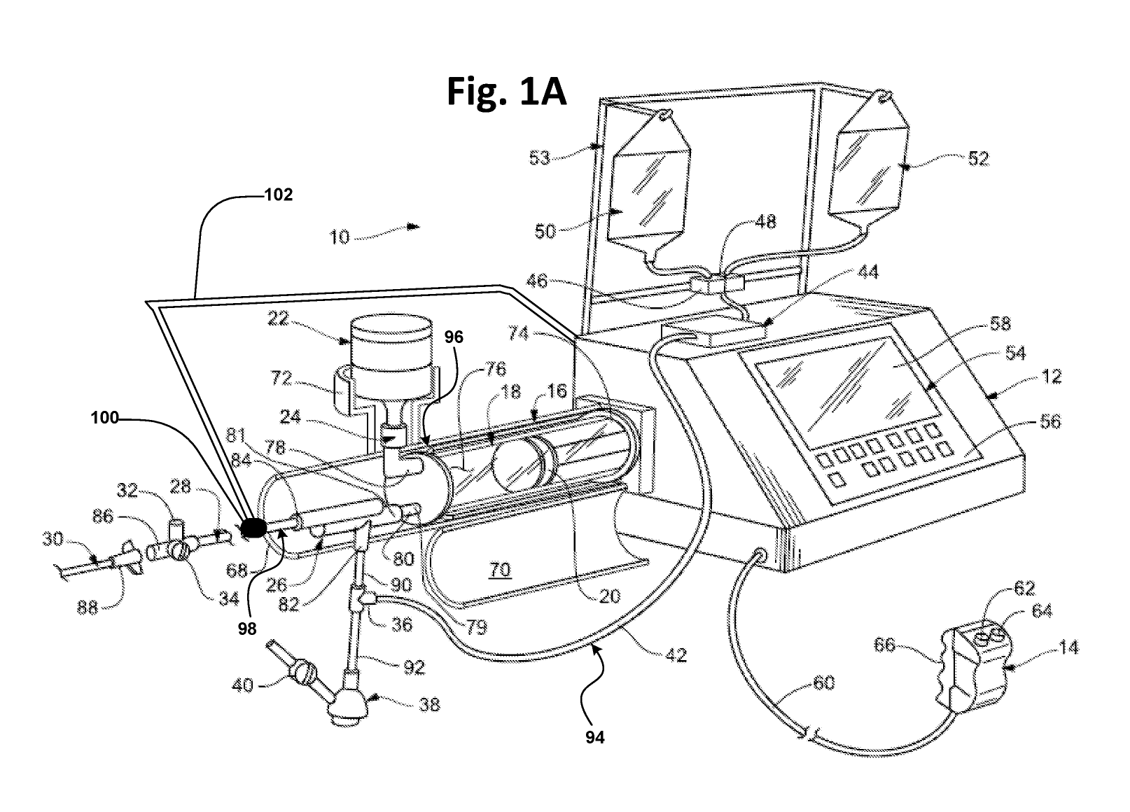

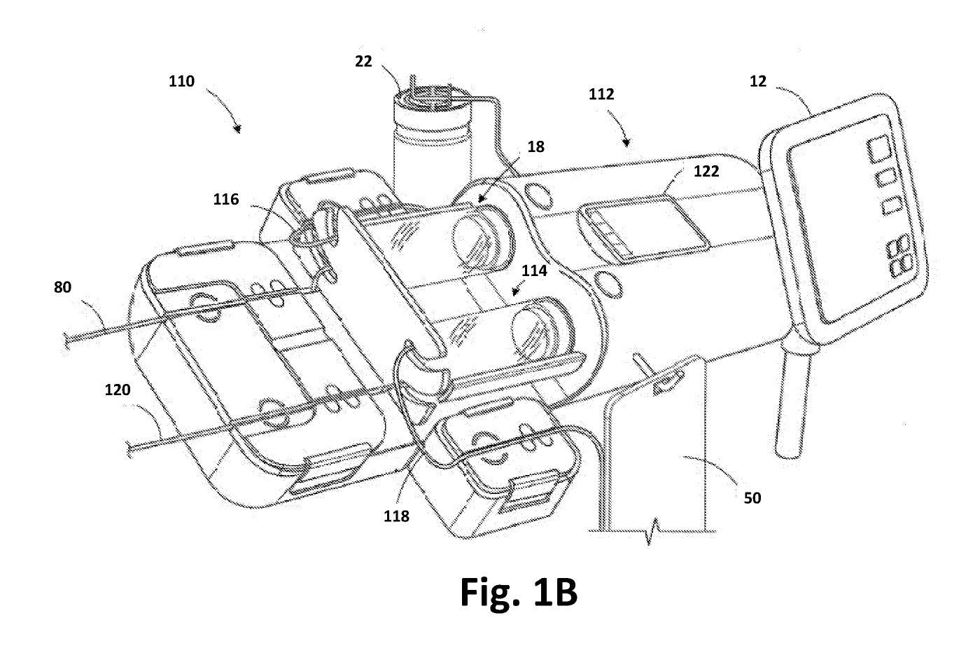

FIG. 1A shows a contrast media injector system 10 for injecting contrast media into a blood vessel under interactive physician control during a medical procedure, such as an angiogram. As shown, system 10 includes main console 12, hand held remote control 14, reservoir holder 16, reservoir main body 18, reservoir plunger/piston 20, radiographic material supply container (“fluid supply container”) 22, inlet valve system 24, manifold 26, high pressure tube 28, catheter 30, patient medication port 32, three-way stop-cock 34, T-connector 36, pressure transducer 38, stop-cock 40, tubing 42, peristaltic pump 44, saline check valve 46, waste check valve 48, saline bag (“fluid supply container”) 50, waste bag 52, and bag support rack 53. It should be noted that the radiographic material supply container 22 and the saline bag 50, along with other kinds of fluid supply containers, may be referred to as fluid supply containers. It should also be noted that system 10 is just one embodiment of a contrast media injector system in accordance with the invention.

In the embodiments shown, console 12 houses the electrical controls for system 10, together with the motors which drive piston/plunger 20 and peristaltic pump 44. On the front surface of console 12, user interface 54 provides control switches 56 and display 58 through which the user may enter control settings and monitor the operational state of system 10.

Remote control 14 can be connected to console 12 by cable 60 (although in other embodiments remote control 14 may be connected by a wireless connection such as an RF, infrared optic, or ultrasonic link). Remote control 14 is, in the embodiment shown in FIG. 1A, a hand-held control which includes reset and saline push button switches 62 and 64, respectively, and flow rate control lever or trigger 66. By applying force to trigger 66, the user can provide a command signal to console 12 to provide a continuously variable injection rate. As will be explained further below, the console 12 can be in communication, such as signal communication, with various components of system 10. In this way, console 12 can be configured to control such components of system 10, which can include controlling operational parameters of one or more components of system 10.

As shown in FIG. 1A, reservoir holder 16 projects from the left hand side of console 12. Reservoir holder 16 is preferably a clear material, and includes a half cylindrical back shell 68, a half cylindrical front door 70 (which is shown in open position in FIG. 1A), and fluid supply container holder 72. The reservoir main body 18 generally includes a transparent or translucent plastic cylinder having its open end 74 connected to console 12. A closed end 76 of reservoir main body 18 contains two ports: inlet port 78 and outlet port 79. Plunger/piston 20 is movable within reservoir main body 18. Plunger/piston 20 can be connected to, and driven by, a motor located within console 12. Reservoir main body 18 can be in signal communication with the console 12 such that control of reservoir main body 18, including components thereof, can be performed at the console 12 (e.g., the console 12 is configured to control one or more operational parameters of the reservoir main body 18, including components thereof).

The contrast fluid supply container 22 is connected through inlet valve system 24 to inlet port 78. Radiographic contrast material is drawn from fluid supply container 22 through inlet valve system 24 and inlet port 78 into the pumping chamber defined by reservoir main body 18 and plunger/piston 20. Inlet valve system 24 is a fluid one-way valve which permits air to flow from reservoir main body 18 back into fluid supply container 22, but will not permit radiographic contrast material to flow from reservoir main body 18 to fluid supply container 22 when fully closed. In one example, the inlet valve system 24, inlet port 78, reservoir main body 18, outlet port 79, and conduit 80 can be embodied as a contrast reservoir kit available as ACIST A2000 from ACIST Medical Systems, Inc., of Eden Prairie, Minn.

In FIG. 1A, the outlet port 79 of reservoir main body 18 is connected to manifold 26 by a conduit 80. Manifold 26 includes a spring biased spool valve which normally connects first fluid inlet (“transducer/saline port”) 82 and fluid outlet (“patient port”) 84. When contrast media is to be injected, the pressure of the contrast media causes the spool valve to change states so that outlet port 79 is connected to patient port 84 via second fluid inlet (“contrast media inlet port”) 81 and conduit 80. Other types of valves that selectively communicate between the contrast media and the saline can be used, including the elastomeric type valves described in Applicant's U.S. Pat. No. 7,617,837. Such a manifold can be used with any contrast injector system, including the CVi contrast injector system offered by ACIST Medical Systems, Inc., of Eden Prairie, Minn. The pertinent parts of U.S. Pat. No. 6,656,157, titled “Infinitely Refillable Syringe,” which describes contrast injector systems, are hereby incorporated by reference.

In the embodiment shown, high pressure tube 28 is a flexible tube which connects patient port 84 to catheter 30. A three-way stop-cock 34 is located at the distal end of tube 28. A rotatable Luer lock connector 86 is connected to stop-cock 34 and mates with Luer connector 88 at the proximal end of catheter 30. A stopcock 34 either blocks flow between tube 28 and catheter 30, permits flow, or connects medication port 32 to catheter 30 (e.g., for use when medication is to be delivered through catheter 30 to the patient).

When catheter 30 is in place in the patient, and an injection of contrast media is not taking place, pressure transducer 38 can monitor the blood pressure through the column of fluid which extends from catheter 30, tube 28, patient port 84, manifold 26, transducer/saline port 82, tubing 90, T-connector 36, and tubing 92. In the embodiment shown, transducer 38 has an associated stop-cock 40 which allows transducer 38 to be exposed to atmospheric pressure during calibration and also allows for removal/expulsion of trapped air so the dome chamber of transducer 38 can be flushed with saline.

Peristaltic pump 44 supplies saline solution from fluid supply container 50 through saline check valve 46, tubing 42, T-connector 36 and tubing 90 to saline port 82. When peristaltic pump 44 is operating to supply saline solution, the saline solution is supplied through manifold 26 to patient port 84 and then through tube 28 to catheter 30. Peristaltic pump 44 also operates in an opposite direction to draw fluid from catheter 30 and through tube 28, manifold 26, tubing 90, T-connector 36 and tubing 42 to waste check valve 48 and then into waste collection bag 52. As mentioned above, saline may be alternatively delivered to the patient with a syringe system instead of a peristaltic pump.

In use, the user (typically a physician) enters into system 10 (e.g., at user interface 54 of console 12) operational parameters (e.g., safety parameters) that will apply to the injection of radiographic contrast material. These parameters typically include the maximum amount of radiographic contrast material to be injected during any one injection, the maximum flow rate of the injection, the maximum pressure developed within reservoir main body 18, and the maximum rise time or acceleration of the injection. To actuate an injection of contrast material, the user operates remote control 14 by squeezing trigger 66. Within the set parameters, system 10 causes the flow rate of the injection to increase as the force or distance of travel of trigger 66 is increased.

For the sake of convenience in describing various embodiments of contrast injector systems (which can use many different components and combinations of such components, including tubing and other fluid communications means), a first fluid communication line 94, a second fluid communication line 96, and a conduit 98 are shown generally in FIG. 1. The first fluid communication line 94 can include any one or more components that serve to define a fluid communication line from the fluid supply container 50 to the first fluid inlet 82 of the manifold 26, even including the fluid supply container 50 and/or first fluid inlet 82 in some cases. As such, the first fluid communication line 94 can include fluid communication means, including combinations of various fluid communication components, extending from the fluid supply container 50 to the first fluid inlet 82 that allow the first fluid inlet 82 to receive fluid from the fluid supply container 50. The particular one or more components that make up the first fluid communication line 94 can vary depending on the specific contrast injector system. In the example of the contrast injector system 10 shown in FIG. 1, the first fluid communication line 94 can include T-connector 36, pressure transducer 38, stop-cock 40, tubing 42, peristaltic pump 44, saline check valve 46, and/or tubing 90, 92. Such an embodiment of the first fluid communication line 94 can be available as ACIST AT-S54 from ACIST Medical Systems, Inc., of Eden Prairie, Minn.

Similarly, the second fluid communication line 96 can include any one or more components that serve to define a fluid communication line from the fluid supply container 22 to the second fluid inlet 81 of the manifold 26, even including the fluid supply container 22 and/or second fluid inlet 81 in some cases. As such, the second fluid communication line 96 can include fluid communication means, including combinations of various fluid communication components, extending from the fluid supply container 22 to the second fluid inlet 81 that allow the second fluid inlet 81 to receive fluid from the fluid supply container 22. The particular one or more components that make up the second fluid communication line 96 can vary depending on the specific contrast injector system. In the example of the contrast injector system 10 shown in FIG. 1, the second fluid communication line 96 can include inlet valve system 24, inlet port 78, reservoir main body 18, outlet port 79, and/or conduit 80. Thus, in the example system 10 of FIG. 1 the reservoir main body 18 is positioned on, and is part of, the second fluid communication line 96.

Furthermore, the conduit 98 can include any one or more components that serve to define a fluid communication line from the fluid outlet 84, of the manifold 26, to a vasculature of a patient through an end of catheter 30, even including the fluid outlet 84 and/or end of catheter 30 in some cases. As such, the conduit 98 can include fluid communication means, including combinations of various fluid communication components, extending from the fluid outlet 84 of the manifold 26 to the end of catheter 30 in the patient that allow the end of catheter 30 in the patient to receive fluid from the fluid outlet 84. The particular one or more components that make up the conduit 98 can vary depending on the specific contrast injector system. In the example of the contrast injector system 10 shown in FIG. 1, the conduit 98 can include high pressure tube 28, three way stopcock 34, connectors 86 and 88, and/or one or all portions of catheter 30.

Contrast injector systems, such as the system 10 shown in FIG. 1A, can include one or more components of the system 10 that are multi-use components used in procedures on different patients (in addition to single-use components used only for a single patient). It is theoretically possible that during a particular procedure one or more multi-use components of a contrast injection system could become exposed to a patient's bodily fluid. If this were to occur, the potential would exist for cross-patient contamination if a previously exposed multi-use component were utilized subsequently with another, different patient. In the embodiment of system 10 shown in FIG. 1A, an example of a multi-use component may be reservoir main body 18 and/or fluid supply container 22. Additional multi-use components may be in some embodiments, for instance, the second fluid communication line 96 (e.g., the inlet valve system 24, inlet port 78, reservoir main body 18, outlet port 79, and/or conduit 80). In the embodiment of system 10 shown in FIG. 1A, one example of a single-use component may be manifold 26. Additional single-use components may be in some embodiments, for instance, the conduit 98 (e.g., the outlet port 79, conduit 80, patient port 84, high pressure tube 28, and/or catheter 30).

To reduce or eliminate risk of cross-patient contamination, contrast injector system embodiments can include one or more devices for addressing potential cross-patient contamination. For example, a sterilization device can be used in embodiments of contrast injector systems to sterilize a fluid path section and thus maintain a sterile barrier between one or more single-use components and one or more multi-use components (e.g., in one application between the conduit 98 and the fluid supply container 22). In the embodiment of system 10 shown in FIG. 1A, a sterilization device 100 (shown schematically in FIG. 1A) is included as one type of device for addressing potential cross-patient contamination.

The sterilization device 100 can be, for instance, any type of component useful for sterilizing a section of a fluid path so as to prevent harmful contaminants from migrating past the sterilization device 100. In some examples, the sterilization device 100 can include an energy emitter for emitting energy appropriate for rendering microorganisms, viruses, or other potentially hazardous contaminants from a patient's vasculature harmless. As one example, the energy emitter of the sterilization device 100 can be an ultraviolet radiation emitter. The ultraviolet radiation emitter of the sterilization device 100 can be configured to emit ultraviolet radiation, including energy in the ultraviolet C (“UVC”) band. The UVC band includes wavelengths between 280 nm and 100 nm. In some examples, the sterilization device 100 may emit UVC radiation at a wavelength of approximately 253 nm to 254 nm, as such UVC radiation may be effective to break molecular bonds of microorganismal DNA and consequently render the microorganism(s) harmless. As will be appreciated, in many embodiments the sterilization device 100, or the system 10 generally, may include means for preventing exposure of those in the vicinity to ultraviolet radiation, such as appropriate insulating means.

As shown in the embodiment of the system 10 in FIG. 1, the sterilization device 100 is positioned on the conduit 98. In particular, the sterilization device 100 is positioned on the conduit 98 such that the energy emitter of the sterilization device 100 is positioned to emit energy (e.g., UVC radiation) to the conduit 98. Therefore, in the illustrated example the sterilization device 100 may serve to sterilize a section of the conduit 98 (which may in some examples be a single-use component). As a result, the sterilization device 100 can act to prevent migration of harmful contaminants past the section of the conduit 98 on which the sterilization device 100 is positioned and in the process maintain a substantially sterile barrier between what can be a single-use component (e.g., conduit 98) and a multi-use component (e.g., reservoir main body 18 and/or fluid supply container 22). As will be described in detail later, the sterilization device 100 can be configured at various other positions in the system 10 to achieve similar benefits.

In the embodiment illustrated in FIG. 1, the sterilization device 100 is in communication with the console 12 via line 102. For instance, the sterilization device 100 can be in signal communication with the console 12 via the line 102 such that the sterilization device 100 is controllable by the console 12. In other embodiments, the sterilization device 100 can be in signal communication with the console 12 via wireless transmission means (e.g., a local area network). Signal communication between the sterilization device 100 and the console 12 can be two-way communication, allowing for the console 12 to send various commands to the sterilization device 100 as well as to receive various signals from the sterilization device 100. In some embodiments, the sterilization device 100 can receive power over the line 102 from the console 12, in addition to signals from the console 12. Thus, the sterilization device 100 can in some embodiments be integrated with the system 10.

Exemplary commands which the console 12 can be configured to communicate to the sterilization device 100 can include a start and/or stop sterilization device command, a change sterilization parameter command, and/or a sterilization status check command. The start and/or stop sterilization device command sent from the console 12 to the sterilization device 100 can act, for instance, to begin and/or end emission of energy from the sterilization device 100, such as turning the energy emitter on and/or off. The change sterilization parameter command sent from the console 12 to the sterilization device 100 can act, for instance, to adjust a wavelength of the energy emitted from the energy emitter of the sterilization device 100 and/or adjust a time duration for which the energy is to be emitted. The sterilization status check command sent from the console 12 to the sterilization device 100 can act, for instance, to solicit a return signal from the sterilization device 100 to the console 12 containing requested data. For example, the console 12 can send a signal to the sterilization device 100 requesting data pertaining to one or more components of the sterilization device 100 (e.g., power supply, energy emitter), and the sterilization device 100 can send a return signal to the console 12 containing data pertaining to one or more components of sterilization device 100 (e.g., status of power supply, status of energy emitter). Thus, in some examples communication from the sterilization device 100 is in response to communication received from the console 12.

In other examples (not shown), the sterilization device 100 can be an in-line accessory to a contrast delivery system. For instance, the sterilization device 100 may not be in communication with the console 12, but rather may have an integrated user interface allowing a user to input commands pertaining to operation of the sterilization device 100 directly to the sterilization device 100. In such examples where the sterilization device 100 is an in-line accessory, the sterilization device may also have a distinct power input separate from the console 12.

FIG. 1B shows another embodiment of a contrast injector system 110 for injecting contrast media or other fluid into a blood vessel under interactive physician control during a medical procedure (e.g., an angiogram). The illustrated system 110 shown here differs from that shown and described with respect to FIG. 1A in that the system 110 utilizes a dual-reservoir injector 112. The first fluid supply container 22 is attached to the injector 112, and the second fluid supply container 50 is also attached to the injector 112. In particular, the first fluid supply container 22 is fluidly connected to the reservoir main body 18 similar to that described with respect to FIG. 1A and the second fluid supply container 50 is fluidly connected to a reservoir main body 114. The reservoir main body 114 can include a plunger similar to that of reservoir main body 18 and can be fluidly connected to the second fluid supply container 50 in a manner that can be similar to that for the reservoir main body 18 and fluid supply container 22.

The injector 112 can operate to draw fluid from fluid supply container 22 into reservoir main body 18 via the line 116 (which can include the inlet valve system 24 and inlet port 78) as well as draw fluid from fluid supply container 50 into reservoir main body 114 via the line 118. The plunger of the reservoir main body 18 can expel fluid, such as pressurized fluid, into conduit 80, and the plunger of the reservoir main body 114 can expel fluid, such as pressurized fluid, into conduit 120. Thus, the system 110 can inject multiple medical fluids through one or more fluid lines or conduits into a patient's vasculature under pressure via operation of the associated plungers.

The system 110 can further include a secondary control panel 122 along with the main console 12 as described with respect to FIG. 1A. Main console 12, and in some embodiments secondary control panel 122, can be in communication with each of reservoir main bodies 18 and 114 so as to set up and control operational parameters of each. The system 110 can include one or more sterilization device 100 (as well as one or more of any other device, such as a vibration device and/or an illuminator device) as described throughout this disclosure. In one example, two sterilization devices 100 can be located respectively on the conduits 80 and 120 and be in communication with the main console 12 (and/or the secondary control panel 122) as described with respect to FIG. 1. In this manner, sterilization devices can operate in association with each of the two reservoir main bodies 18, 144.

For purposes of illustration, general representative operations of system 10 will now be described, including contrast fill, air purge, and patient inject operations. Of course, system 10 can also be configured to perform many other types of operations including, for example, saline flush and patient pressure monitoring operations. Although the system 10 is described here, any of the operations and features described can also be used with the system 110 of FIG. 1B.

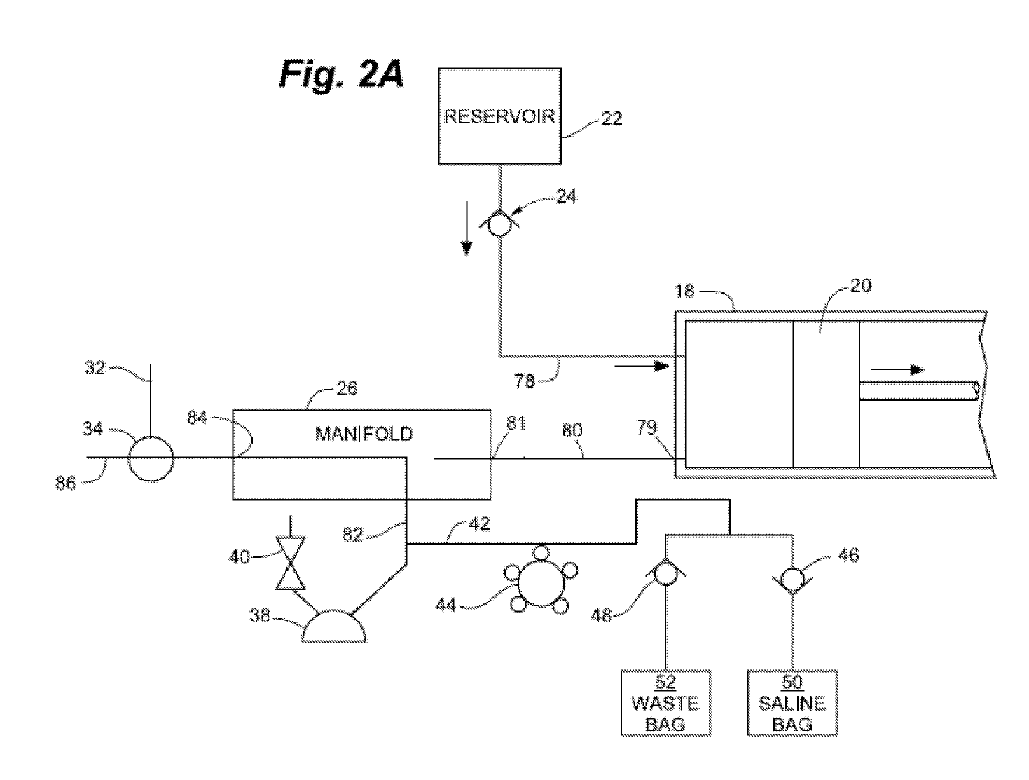

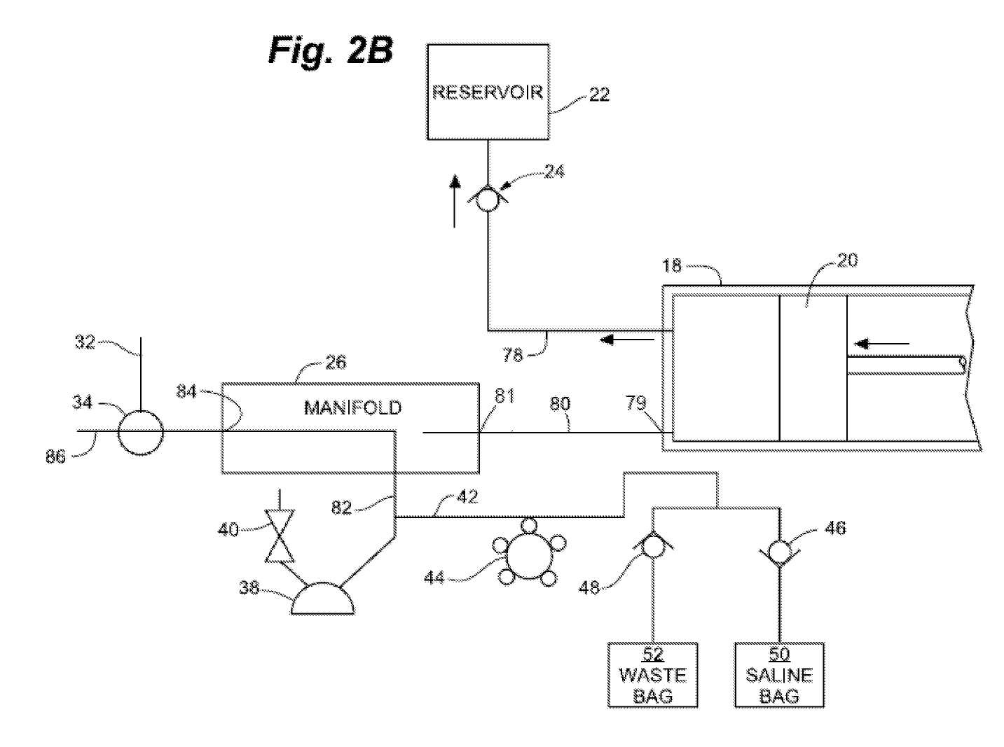

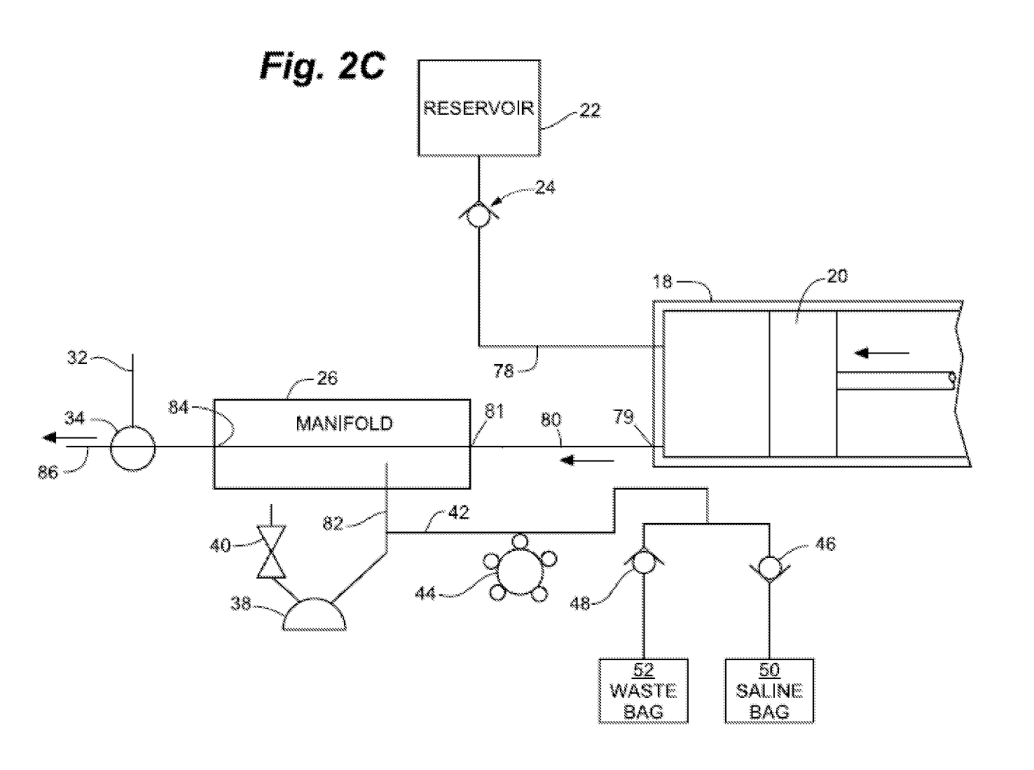

The contrast fill operation illustrated in FIG. 2A involves the filling of reservoir main body 18 with contrast media from fluid (e.g., contrast media) supply container 22. The contrast fill operation can be performed during initial set up of system 10, and may be repeated during operation of system 10 whenever reservoir main body 18 is running low on radiographic contrast material. During initial set up of the system, plunger/piston 20 is initially driven to its furthest forward position adjacent closed end of reservoir main body 18. This will expel to the atmosphere the majority of the air which is located within reservoir main body 18.

Plunger/piston 20 is then retracted, which creates a vacuum within reservoir main body 18 which draws contrast material from fluid supply container 22 through inlet valve system 24 into reservoir main body 18 through inlet port 78.

The contrast fill operation typically will result in some air being drawn into or remaining within reservoir main body 18. It is important, of course, to prevent air from being injected into the patient through catheter 30. The location of two ports at different elevations allows for a greater amount of safety in preventing air bubbles in the injection. Further, in some embodiments, the reservoir can be placed at an angle relative to horizontal (e.g., about 10 degrees from horizontal), such that its closed end, and inlet port 78, are at a higher elevation than its open end. Such an embodiment facilitates air removal from the reservoir through inlet port 78.

During the air purge operation, as illustrated in FIG. 2B, plunger/piston 20 travels forward to expel trapped air within reservoir main body 18. The air, being lighter than the contrast media, gathers near the top of reservoir main body 18. As plunger/piston 20 moves forward, the air is expelled from reservoir main body 18 through inlet port 78 and inlet valve system 24. In the embodiment illustrated in FIG. 2B, inlet valve system 24 allows flow of contrast media from fluid supply container 22 to inlet port 78, but will not allow contrast media to flow in the opposite direction from inlet port 78 to fluid supply container 22. Inlet valve system 24 will, however, allow air to flow from port 78 to fluid supply container 22 until sufficient pressure builds in the reservoir to close the inlet valve system.

FIG. 2C illustrates a patient inject operation. In this operation, plunger/piston 20 travels forward under the interactive control of the user, who may be controlling trigger 66 of remote control 14. The movement of plunger/piston 20 creates hydraulic pressure to force contrast material out of reservoir main body 18 through outlet port 79 and through manifold 26 and high pressure tube 28 into catheter 30. As shown in FIG. 2C, reservoir outlet port 79 and patient port 84 are connected via contrast media inlet port 81 and conduit 80 for fluid flow during the patient inject operation.

In the embodiments shown, manifold 26 contains a valve which controls the routing of fluid connections between patient port 84 and either reservoir outlet port 79 or transducer/saline port 82. As shown, manifold 26 can include a spool valve which is spring biased so that patient port 84 is normally connected to transducer/saline port 82 (as illustrated in FIGS. 2A and 2B). When the pressure at reservoir outlet port 79 builds with the movement of plunger/piston 20 forward, the bias force against the spool valve is overcome so that reservoir outlet port 79 is connected to patient port 84, and transducer/saline port 82 is disconnected. The valve within manifold 26 can protect pressure transducer 38 from being exposed to the high pressure generated by the patient inject operation. The spool valve opens automatically during the patient inject operation in response to increase pressure exerted on it from the reservoir outlet port 79. The spool valve closes and returns to its original position allowing for connection of patient port 84 to transducer 38 when a slight vacuum is applied by retraction of plunger/piston 20 at the end of each patient inject operation. In an alternative embodiment, the valve within manifold 26 is an electromechanical or motor driven valve which is actuated at appropriate times to connect either reservoir outlet port 79 or transducer/saline port 82 to patient port 84. In such embodiments, the valve, and thus actuator mechanism, can be controlled by console 12. Once again, in this alternative embodiment, the valve protects pressure transducer 38 from being exposed to high pressure.

The operation of the contrast injector system can be controlled by any suitable method. In general, the controls will include a digital computer which receives input signals from remote control 14 and front panel controls 56, and provides signals to display 58 to display operation data, alerts, status information and operator prompts, and controls the motion of plunger/piston 20 through a motor drive circuit with a motor.

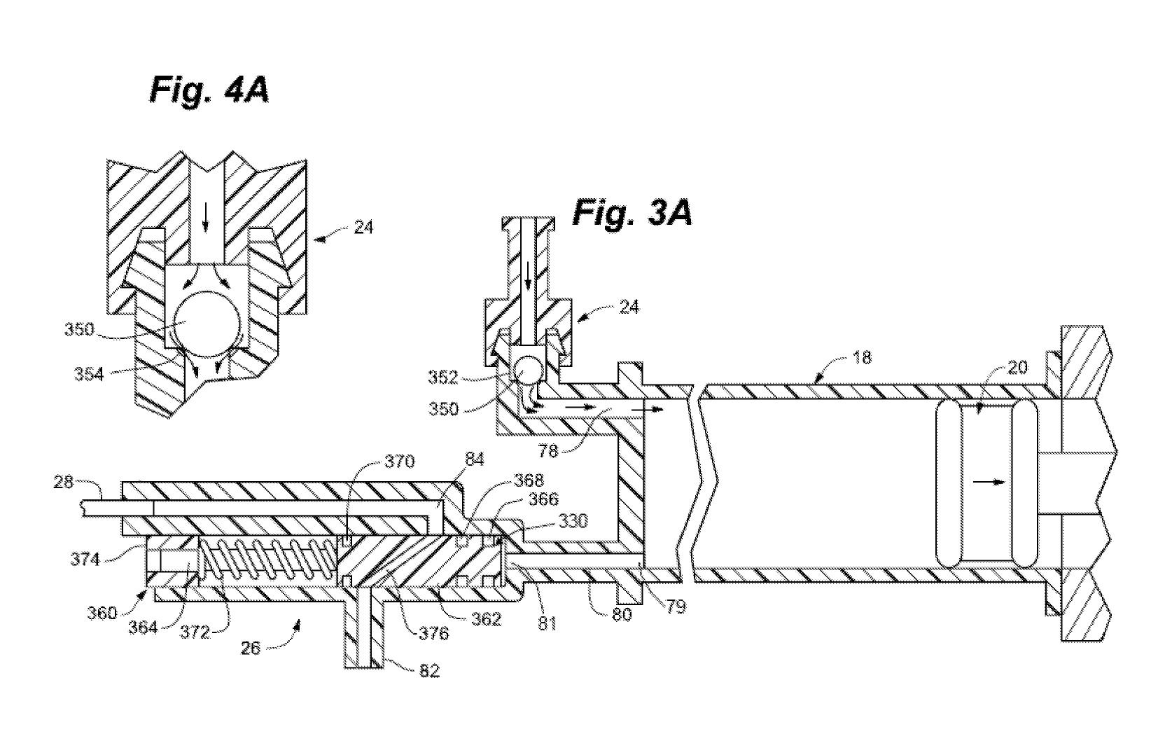

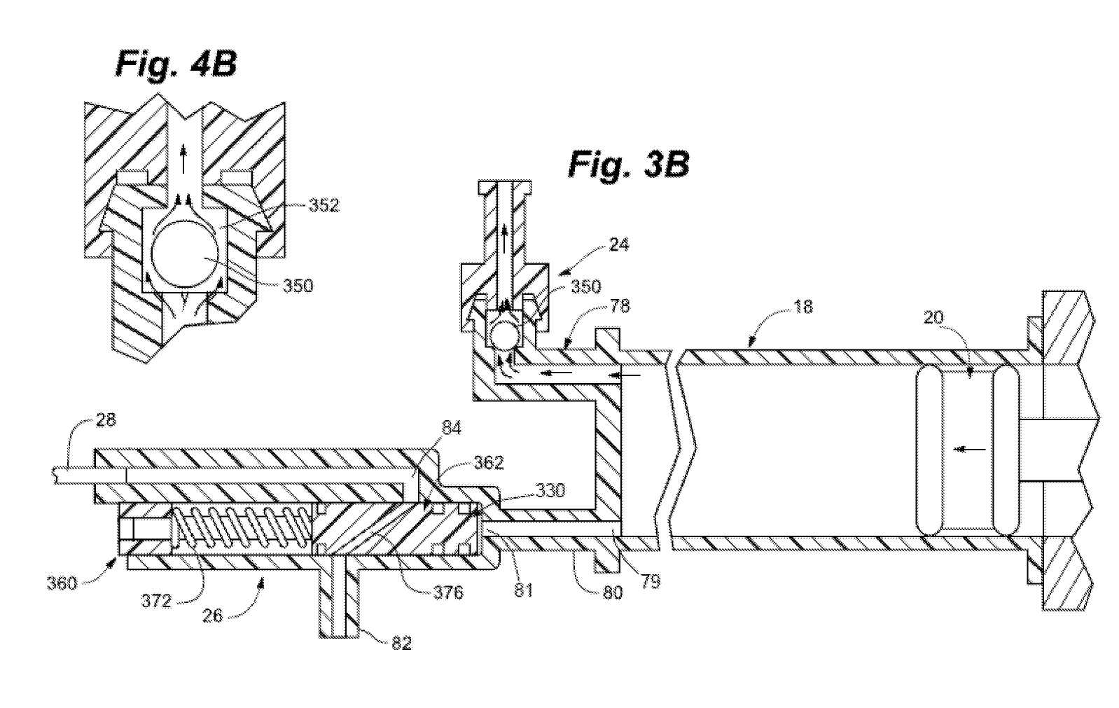

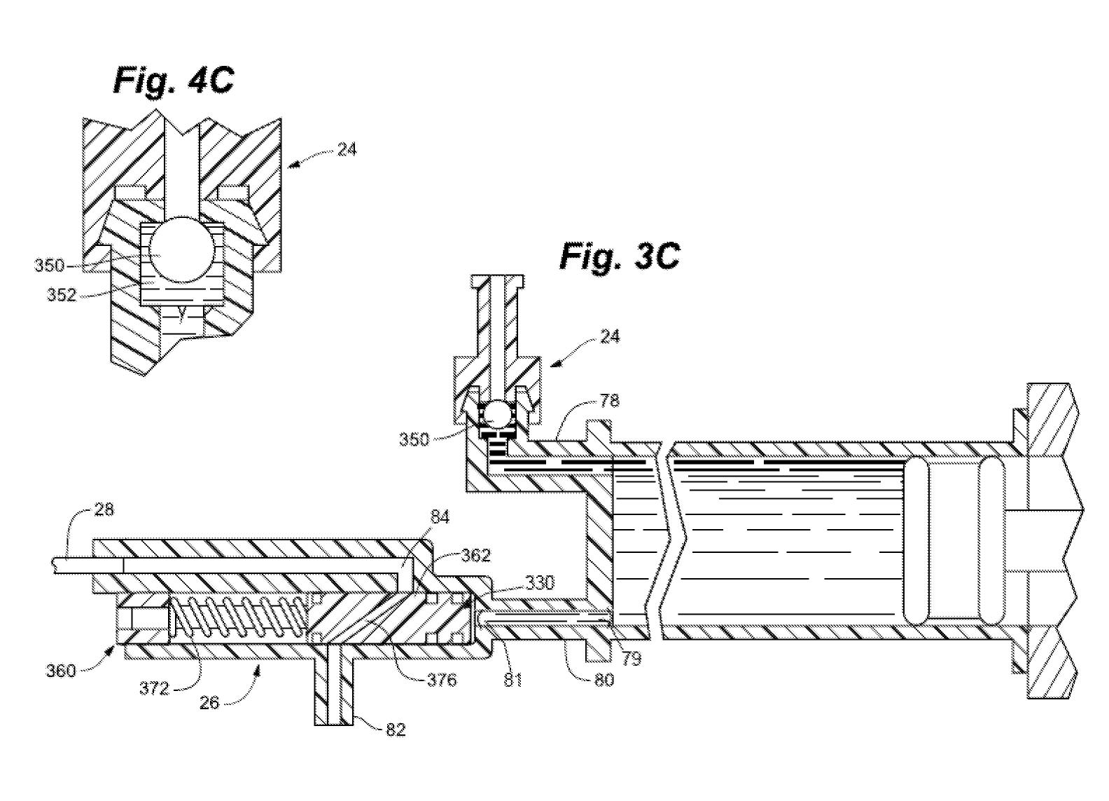

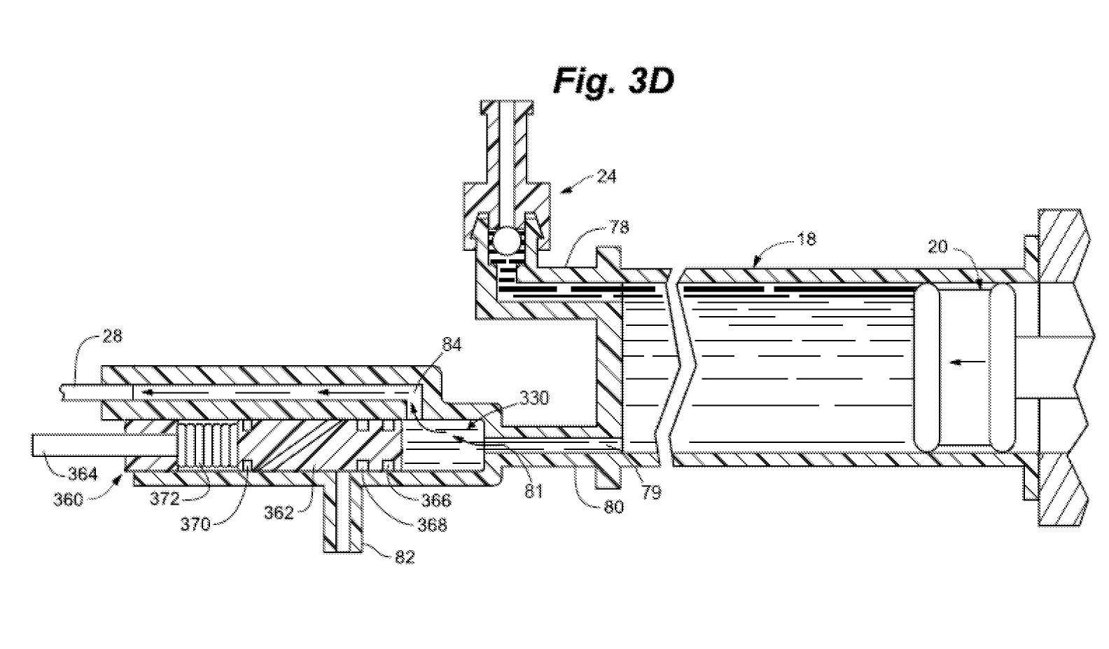

FIGS. 3A-3D and 4A-4C illustrate the general operation of an embodiment of an inlet valve system 24 and manifold 26 during contrast fill, air purge and patient injection operations.

FIGS. 3A and 4A illustrate an embodiment of an inlet valve system 24, manifold 26, reservoir main body 18, and plunger/piston 20 during a contrast fill operation. As shown, inlet valve system 24 includes a valve member 350 which is positioned at a lower seated position within valve chamber 352 in FIGS. 3A and 4B. For purposes of illustration, valve member 350 is represented as a ball in FIGS. 3A-4C. However, valve member 350 may include a wide variety of shapes and features. As shown, contrast media is being drawn into reservoir main body 18 by the rearward movement of plunger/piston 20. The contrast material flows through passages 354 around valve member 350 and into inlet port 78.

As shown, manifold 26 contains main passageway 330, which includes a valve (“spring loaded spool valve”) 360. Furthermore, spring loaded spool valve 360 includes spool body 362, shaft 364, O-rings 366, 368 and 370, bias spring 372, and retainer 374. As shown in FIG. 3A, during the contrast fill operation, bias spring 372 urges spool body 362 to its right-most position toward reservoir main body 18. In this position, spool body 362 blocks outlet port 79 of reservoir main body 18 while connecting transducer saline port 82 to patient port 84 through diagonal passage 376. O-rings 366 and 368 on the one hand, and O-ring 370 on the other hand, are positioned on the opposite sides of diagonal passage 376 to provide a fluid seal.

FIGS. 3B and 4B illustrate an embodiment of an air purge operation. Reservoir main body 18 has been filled with contrast fluid, but also contains trapped air. Plunger/piston 20 is driven forward to force the air out of reservoir main body 18 through inlet port 78 and through inlet valve system 24 around the valve member. During the air purge operation, spool valve 360 is in the same position as in FIG. 3A. Diagonal passage 376 connects transducer saline port 82 with patient port 84. As a result, pressure monitoring by pressure transducer 38 can be performed during the air purge (as well as the contrast fill) operation.

FIGS. 3C and 4C illustrate the state of manifold 26 and inlet valve system 24 at the end of the air purge operation and at the beginning of a patient inject operation. In FIG. 3C, all air has been expelled from reservoir main body 18. Valve member 350 may float on the radiographic contrast material, so that when all air has been removed and the radiographic contrast material begins to flow out of reservoir main body 18 and through inlet port 78 to valve chamber 352, valve member 350 is moved upwards to its upper seated position. Valve member 350 blocks any continued upward flow of contrast media, as is illustrated in FIGS. 3C and 4C.

In the state which is illustrated in FIG. 3C, the pressure within reservoir main body 18, and specifically the pressure in outlet port 79, has not yet reached a level at which the bias force of spring 372 has been overcome. As a result, spool body 362 has not yet moved to the left and diagonal passage 376 continues to connect transducer saline port 82 with patient port 84.

FIG. 3D illustrates an embodiment of a patient inject operation. Plunger/piston 20 is moving forward, and inlet valve system 24 is closed. The pressure at outlet port 79 has become sufficiently high to overcome the bias force of spring 372. Spool body 362 has been driven to the left so that outlet port 79 is connected to patient port 84 through main passageway 330. At the same time, spool body 362 blocks transducer/saline port 82. By virtue of the operation of spool valve 360, the high pressure generated by movement of plunger/piston 20 and reservoir main body 18 is directly connected to patient port 84, while saline port 82 and pressure transducer 38 are protected from the high pressure. The pressure to actuate may be variable and determined after manufacture by increasing or decreasing the reservoir preload.

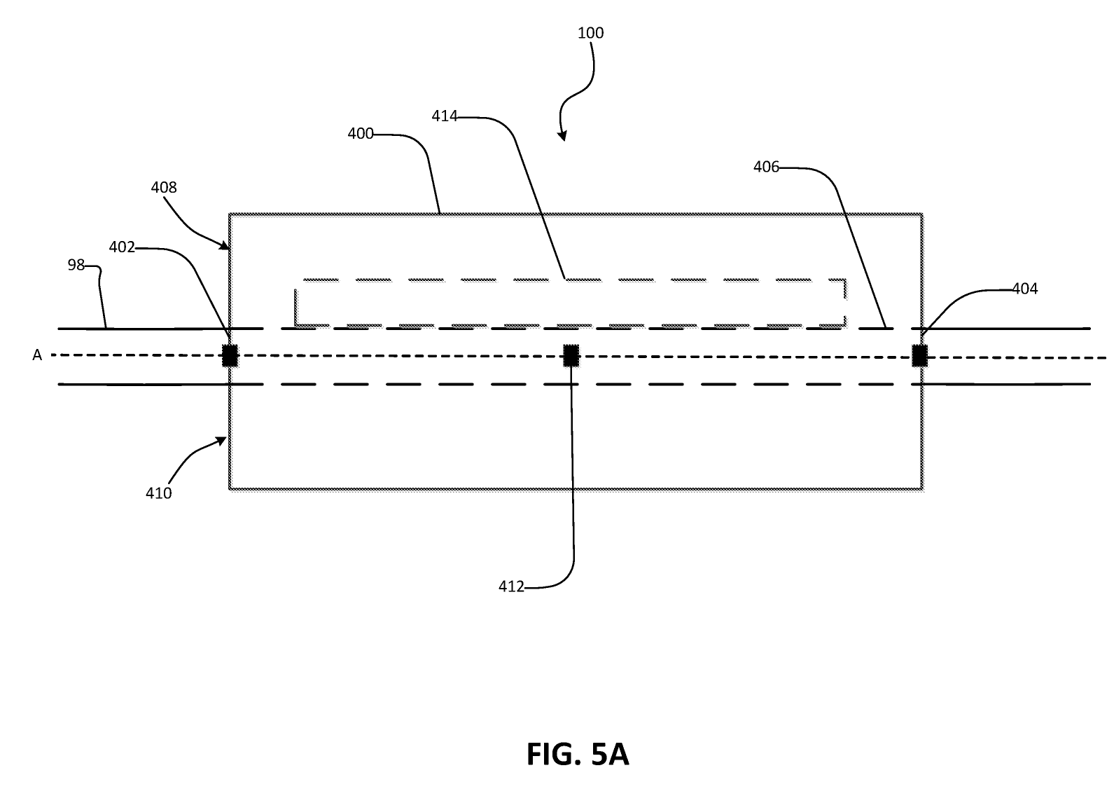

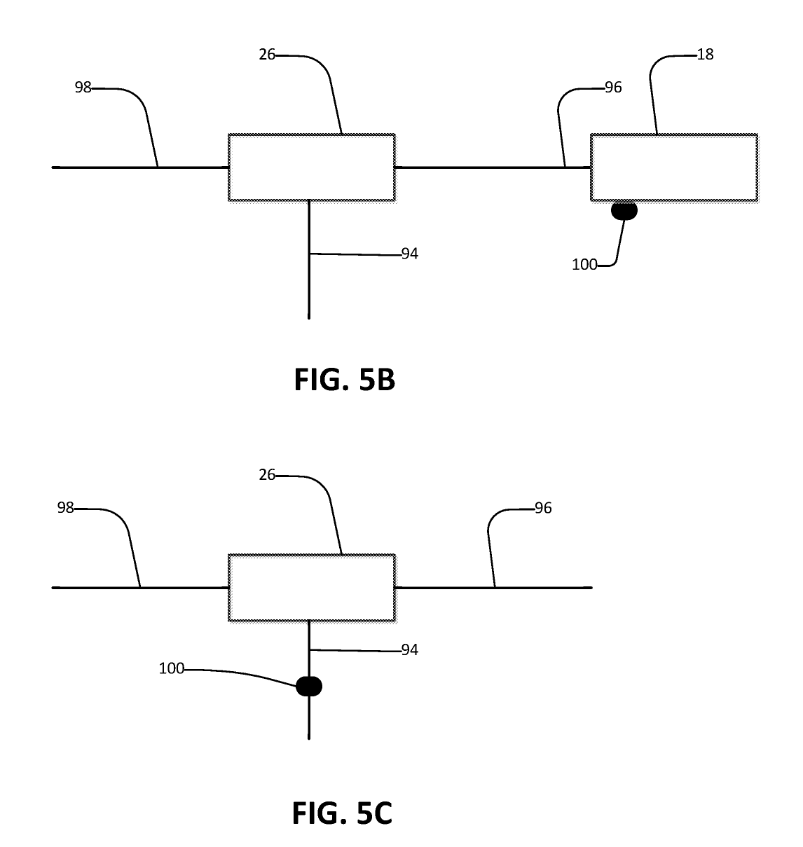

FIG. 5A is a schematic, side elevational diagram of an embodiment of the sterilization device 100. The sterilization device 100 can include a housing 400 defining a housing inlet 402, a housing outlet 404, and a housing channel 406 extending within the housing 400 from the housing inlet 402 to the housing outlet 404. The housing channel 406 can be configured to receive tubing or other fluid communication means through which fluid is communicated during use of a contrast injector system, such that the housing channel 406 may act to hold the portion of the tubing or other fluid communication means extending through the housing 400. In other examples, the housing inlet 402 can connect to an end of tubing or other fluid communication means so as to receive fluid from the end of the tubing or other fluid communication means and pass this received fluid into the housing channel 406. In such examples, this fluid can be communicated directly through the housing channel 406 to the housing outlet 404, which may be connected to an end of tubing or other fluid communication means acting to receive the fluid from the housing channel 406 via the housing outlet 404.

In the illustrated example of FIG. 5A, the housing 400 receives and holds a portion of tubing or other fluid communication means within the housing channel 406. As described and shown with respect to FIG. 1 previously, the sterilization device 100 can be positioned in one example on the conduit 98 such that a portion of the conduit 98 extends through the housing 400 via the housing channel 406. The housing 400 as shown can include a first housing portion 408 and a second housing portion 410. In the example show in FIG. 5A, the first housing portion 408 includes a top portion of the housing 400, while the second housing portion 410 includes a bottom portion of the housing 400, but in other embodiments the first and second housing portion 408, 410 can include other portions of the housing 400 as desired.

One or both of the first and second housing portions 408, 410 can be movable, for instance to facilitate access to the housing channel 406. In one example, the sterilization device 100 may include one or more hinges 412 connecting the first and second housing portion 408, 410, and thus configuring the first and/or second housing portions 408, 410 to be movable, such as relative to one another about an axis A. For instance, when the second housing portion 410 is unsecured from the first housing portion 408 the hinges 412 can allow the second housing portion 410 to pivot about the first housing portion 408 at axis A, for example about one hundred and eighty degrees so as to be adjacent to (rather than below) the first housing portion 408. Moving the second housing portion 410 can allow for access to an axial length of at least a portion of the housing channel 406 (a portion of the housing channel 406 may be on the movable second housing portion 410 in some embodiments), and thus facilitate positioning the sterilization device 100 on the conduit 98 (or tubing or other fluid communication means). Once the sterilization device 100 has been positioned on the conduit 98 (or tubing or other fluid communication means) so as to receive the conduit 98 within the housing channel 406, the first and second housing portions 408, 410 can be secured together similar to that shown in FIG. 5A.

As also described previously with respect to FIG. 1, the sterilization device 100 can include an energy emitter 414, such as an ultraviolet radiation emitter. In embodiments where the energy emitter 414 is an ultraviolet radiation emitter, sources of radiation, including UVC radiation, emitted from the energy emitter 414 can include a mercury vapor lamp, xenon iodide excimer (e.g., exciplex) lamp, and/or krypton fluoride excimer (e.g., exciplex) laser. In other embodiments, the energy emitter 414 can include an ultrasonic transducer configured to emit ultrasound waves into the conduit 98 (or tubing or other fluid communication means). In some such embodiments, the ultrasonic transducer can be further configured to focus the emitted ultrasound waves so as to produce destructive ultrasound waves capable of rendering contaminants harmless.

As shown in FIG. 5A, the energy emitter 414 can be located within the housing 400 and positioned to emit energy to the conduit 98. In particular, in some embodiments, such as that shown in FIG. 5A, the energy emitter 414 can be located within the housing 400 at a region interfacing with (e.g., contacting) the housing channel 406. In this way, when the conduit 98 is received within the housing channel 406 the conduit 98 interfaces with (e.g., contacts) the energy emitter 414. In some examples, the energy emitter 414 within the housing 400 can have an axial length greater than half of the axial length of the housing 400, such as an axial length greater than seventy five percent of the axial length of the housing 400 or greater than ninety percent of the axial length of the housing 400. The energy emitter 414 can be configured to emit energy from one axial side (e.g., an axial side interfacing with the housing channel 406 and thus conduit 98) at substantially all locations along an axial length of this side such that the longer the axial length of the energy emitter 414 within the housing 400, the longer period of time during which the fluid traveling through conduit 98 is exposed to the energy emitted by the energy emitter 414. Such energy emitters may lead to more effective sterilization. Also within the housing 400 can be insulating material (not shown) configured to prevent energy emitted from the energy emitter 414 from escaping the housing 400. Various other configurations of the housing 400 can also be used, with various positions of internal components as desired. In further embodiments, a sterilization device can include a component, such as integrated with the energy emitter, to provide quantitative assessment of contaminant presence. For instance, such component of the sterilization device may provide a measurement of a contaminant density value at the location where the sterilization device is positioned. This quantitative data can be communicated to and used by the console in operation of the system.

FIGS. 5B and 5C are schematic diagrams illustrating portions of contrast injector system embodiments having differing locations of the sterilization device 100 other than on the conduit 98 as in FIG. 5A. FIG. 5B shows the sterilization device 100 positioned on the second fluid communication line 96. One exemplary position of the sterilization device 100 on the second fluid communication line 96, as shown in FIG. 5B, can be on the reservoir main body 18. In such a location, the sterilization device 100 can act to sterilize the contents of the reservoir main body 18. In this example, the sterilization device may be sized so as to be large enough to encompass the reservoir main body 18 as with the conduit 98 in FIG. 5A or the sterilization device can have a different configuration allowing the energy emitter of the sterilization device to interface with the surface of the reservoir main body 18. Another exemplary position of the sterilization device 100 on the second fluid communication line 96 can be upstream of the reservoir main body 18 (e.g., between the reservoir main body 18 and the manifold 26). As such, the energy emitter of the sterilization device 100 can be positioned to emit energy to the second fluid communication line 96. FIG. 5C shows the sterilization device 100 positioned on the first fluid communication line 94. As such, the energy emitter of the sterilization device 100 can be positioned to emit energy to the first fluid communication line 94. The sterilization device shown in the positions in FIGS. 5B and 5C can be configured similar, including configuration of internal components, to that described and shown with respect to FIG. 5A, except that the conduit 98 in FIG. 5A can be the first or second fluid communication line 94 or 96 in FIG. 5C or FIG. 5B, respectively.

In some examples, a first sterilization device 100 can be positioned on the first fluid communication line 94 and a second sterilization device 100 can be positioned on the second fluid communication line 96. In further examples, a first sterilization device 100 can be positioned on the first fluid communication line 94, a second sterilization device 100 can be positioned on the second fluid communication line 96, and a third sterilization device 100 can be positioned on the conduit 98. In addition, the sterilization device(s) 100 can be in communication with the console as detailed above. In examples utilizing more than one sterilization device 100 at differing locations, the console can be in independent and/or synchronized communication with each sterilization device 100. This can allow for independent and/or synchronized control over each sterilization device 100. The portions of the contrast injector system embodiments shown in FIGS. 5A-5C can be included in the contrast injector system embodiments shown and described previously.

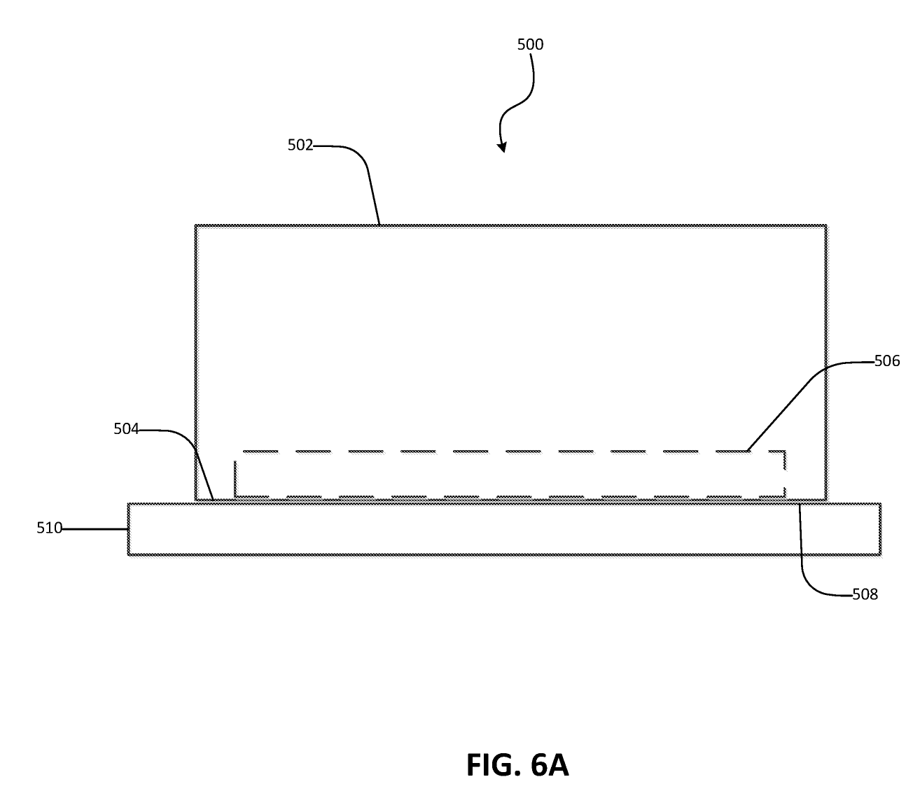

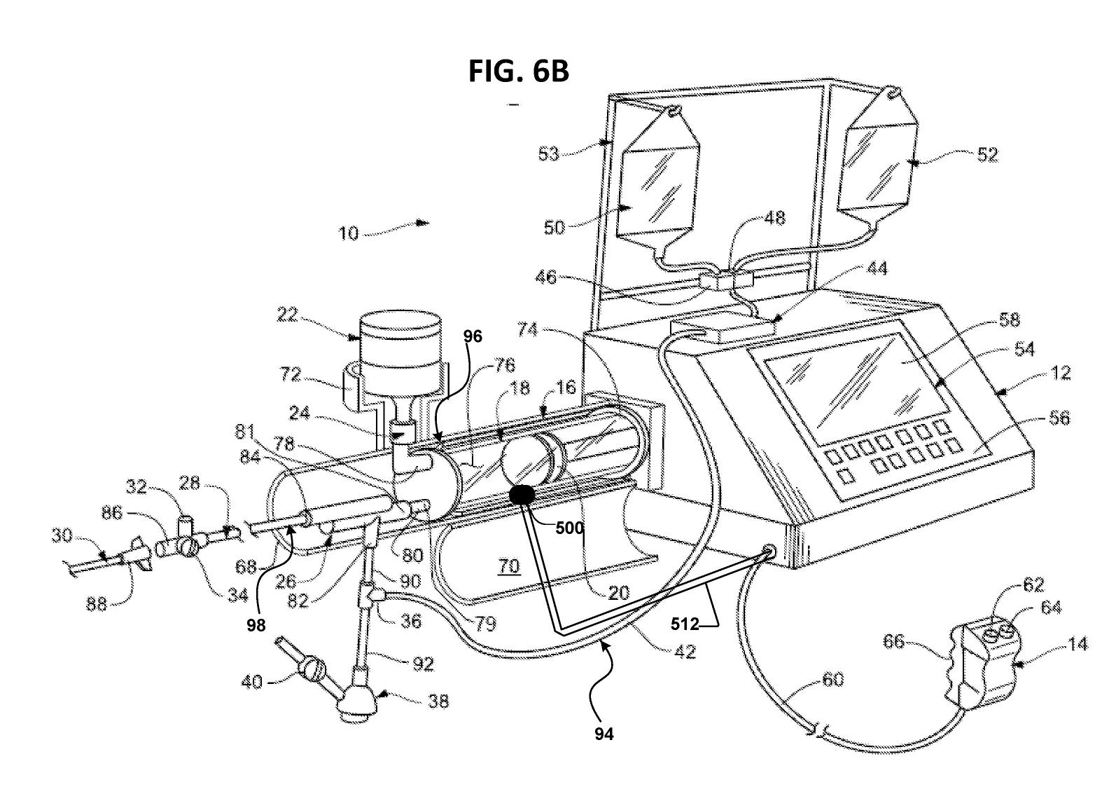

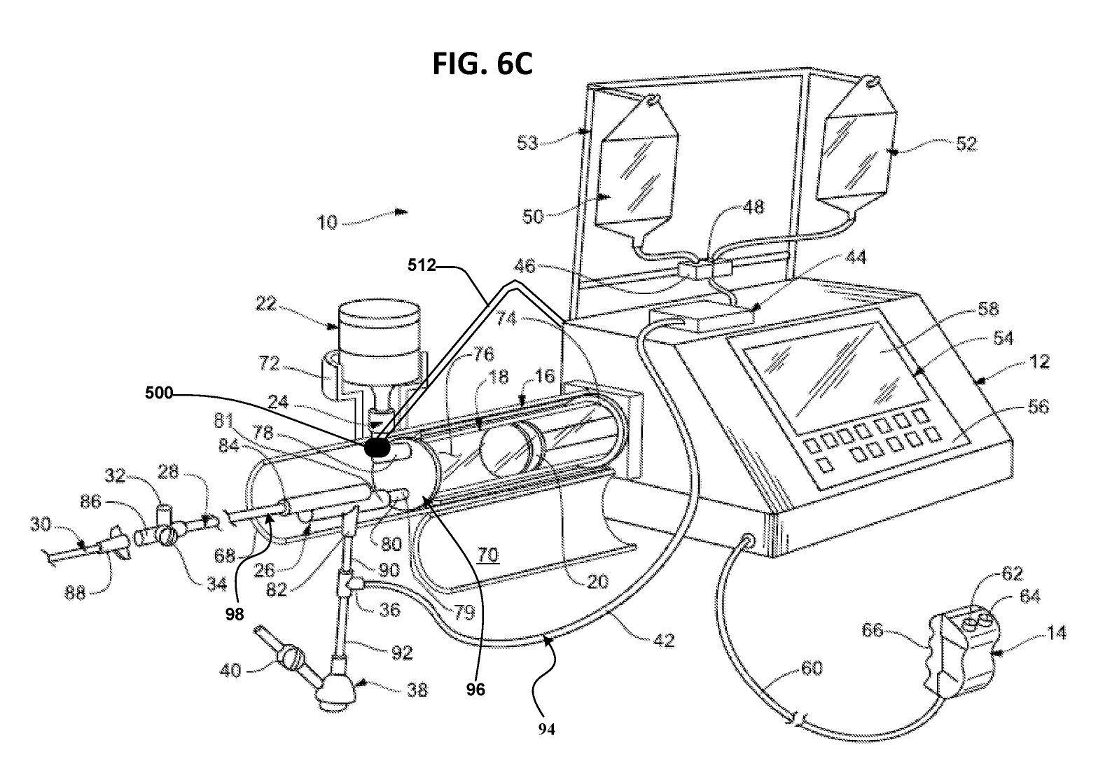

In addition to, or as an alternative to, use of sterilization device(s) as described, other devices can be included in embodiments of a contrast injector system to address potential cross-patient contamination. FIGS. 6A-6C illustrate use of a vibration device in embodiments of a contrast injector system.

FIG. 6A shows a schematic diagram of an embodiment of a vibration device 500. The vibration device 500 can be any device capable of inducing acoustic vibrations onto an interfacing component surface. The exemplary vibration device 500 shown can include a housing 502 having a first housing surface 504. Disposed within the housing 502 can be a piezoelectric actuator 506. The piezoelectric actuator 506 may be a low-frequency actuator configured to operate at a frequency between approximately 100 kHz to 300 kHz. As shown in the illustrated example, the piezoelectric actuator 506 can be located within the housing 502 so as to be proximate to a surface 508 of an interfacing component 510. In particular, where the first housing surface 504 interfaces (e.g., is in apposition) with the surface 508 of the interfacing component 510, the piezoelectric actuator 506 can be disposed on (e.g., contact) the first housing surface 504 as shown in FIG. 6A. Such a configuration can allow acoustic vibrations to be imparted from the piezoelectric actuator 506 directly onto the surface 508 of the interfacing component 510. Imparting acoustic vibrations onto the surface 508 of the interfacing component 510 can help to reduce or eliminate formation of contaminants (e.g., various biofilm formations) within the interfacing component 510, such as along the surface 508 of the interfacing component 510. In some embodiments, the vibration device can include an ultrasonic transducer configured to emit ultrasound waves into the interfacing component 510 in addition to, or as an alternative to, the piezoelectric actuator. In some such embodiments, this ultrasonic transducer can be further configured to focus the emitted ultrasound waves so as to produce destructive ultrasound waves capable of rendering contaminants harmless.

FIG. 6B shows the embodiment of the system 10 described previously, in which like numbers represent like features. In FIG. 6B the vibration device 500 (shown schematically in FIG. 6B) is included in the system 10. In the illustrated example, the vibration device 500 is disposed on the second fluid communication line 96, and specifically here on a surface of the reservoir main body 18, such that the surface of the reservoir main body 18 is the surface of the interfacing component described in FIG. 6A. Thus, the piezoelectric actuator of the vibration device 500 can contact, via the housing surface, the surface of the reservoir main body 18 to impart acoustic vibrations directly thereon to reduce or eliminate formation of contaminants (e.g., various biofilm formations) within the reservoir main body 18. In another example, the vibration device 500 may be disposed on the second fluid communication line at a surface of the fluid supply container 22 (e.g., contrast media supply container), such that the surface of the fluid supply container 22 is the surface of the interfacing component described in FIG. 6A. Thus, the piezoelectric actuator of the vibration device 500 can contact, via the housing surface, the surface of the fluid supply container 22 to impart acoustic vibrations directly thereon to reduce or eliminate formation of contaminants within the fluid supply container 22. In certain embodiment, two vibration devices 500 can be included in the system 10 on the surface of the reservoir main body 18 and the surface of the fluid supply container 22. In some embodiments, one or more of each of the vibration devices 500 and sterilization devices can be used in a single contrast injector system.

FIG. 6C also shows the embodiment of the system 10 described previously, in which like numbers represent like features and which includes the vibration device 500 (shown schematically in FIG. 6C). In the example of FIG. 6C, the vibration device 500 is disposed on a portion of the second fluid communication line 96, such that a surface of the second fluid communication line 96 is the surface of the interfacing component described in FIG. 6A. In particular, the vibration device 500 is disposed on the second fluid communication line 96 at a portion of the second fluid communication line 96 having inlet port 78, and thus between the fluid supply container 22 and the reservoir main body 18. Thus, the piezoelectric actuator of the vibration device 500 can contact, via the housing surface, the surface of the second fluid communication line 96 to impart acoustic vibrations directly thereon to reduce or eliminate formation of contaminants (e.g., various biofilm formations) within the second fluid communication line 96. In one example, a first vibration device 500 can be disposed on a surface of the fluid supply container 22 and a second vibration device 500 can be disposed on a portion of the second fluid communication line 96 (or any other fluid communication line or conduit). In another example, a first vibration device 500 can be disposed on a surface of the reservoir main body 18 and a second vibration device 500 can be disposed on a portion of the second fluid communication line 96 (or any other fluid communication line or conduit). As noted, in some embodiments, one or more vibration devices 500 and one or more sterilization devices can be used in a single contrast injector system in various locations, such as those described herein.

As also shown in the examples of FIGS. 6B and 6C, the vibration device 500 is in communication with the console 12 via line 512. For instance, the vibration device 500 can be in signal communication with the console 12 via the line 512 such that the vibration device 500 is controllable by the console 12. In other embodiments, the vibration device 500 can be in signal communication with the console 12 via wireless transmission means (e.g., a local area network). Signal communication between the vibration device 500 and the console 12 can be two-way communication, allowing for the console 12 to send various commands to the vibration device 500 as well as to receive various signals from the vibration device 500. In some embodiments, the vibration device 500 can receive power over the line 512 from the console 12, in addition to signals from the console 12. Thus, the vibration device 500 can in some embodiments be integrated with the system 10.

Exemplary commands which the console 12 can be configured to communicate to the vibration device 500 can include a start and/or stop vibration command, a change vibration parameter command, and/or a vibration status check command. The start and/or stop vibration command sent from the console 12 to the vibration device 500 can act, for instance, to begin and/or end generation of acoustic vibrations by the piezoelectric actuator, such as turning the piezoelectric actuator on and/or off. The change vibration parameter command sent from the console 12 to the vibration device 500 can act, for instance, to adjust an operational frequency of the piezoelectric actuator and/or adjust a time duration for which the piezoelectric actuator is to operate. The vibration status check command sent from the console 12 to the vibration device 500 can act, for instance, to solicit a return signal from the vibration device 500 to the console 12 containing requested data. For example, the console 12 can send a signal to the vibration device 500 requesting data pertaining to one or more components of the vibration device 500 (e.g., power supply, piezoelectric actuator), and the vibration device 500 can send a return signal to the console 12 containing data pertaining to one or more components of the vibration device 500 (e.g., status of power supply, status of piezoelectric actuator). Thus, in some examples communication from the vibration device 500 is in response to communication received from the console 12. Where more than one vibration device 500 is utilized, the console 12 can be independent and/or synchronized communication with each of the vibrations devices 500.

In other examples (not shown), the vibration device 500 can be an in-line accessory to a contrast delivery system. For instance, the vibration device 500 may not be in communication with the console 12, but rather may have an integrated user interface allowing a user to input commands pertaining to operation of the vibration device 500 directly to the vibration device 500. In such examples where the vibration device 500 is an in-line accessory, the vibration device may also have a distinct power input separate from the console 12.

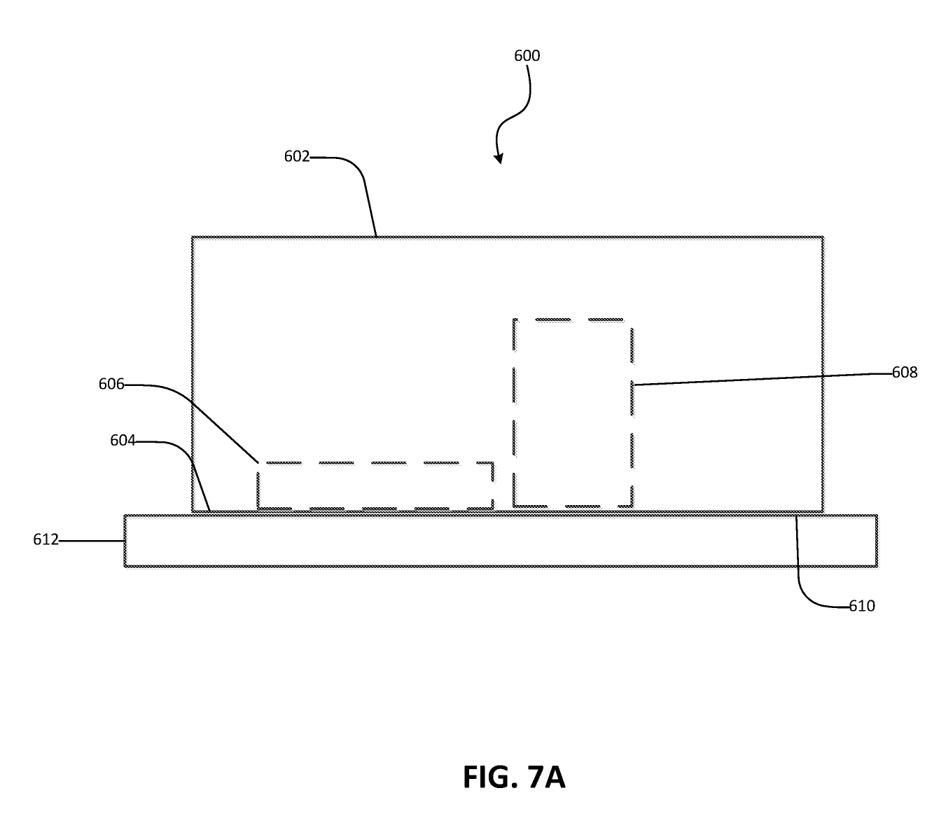

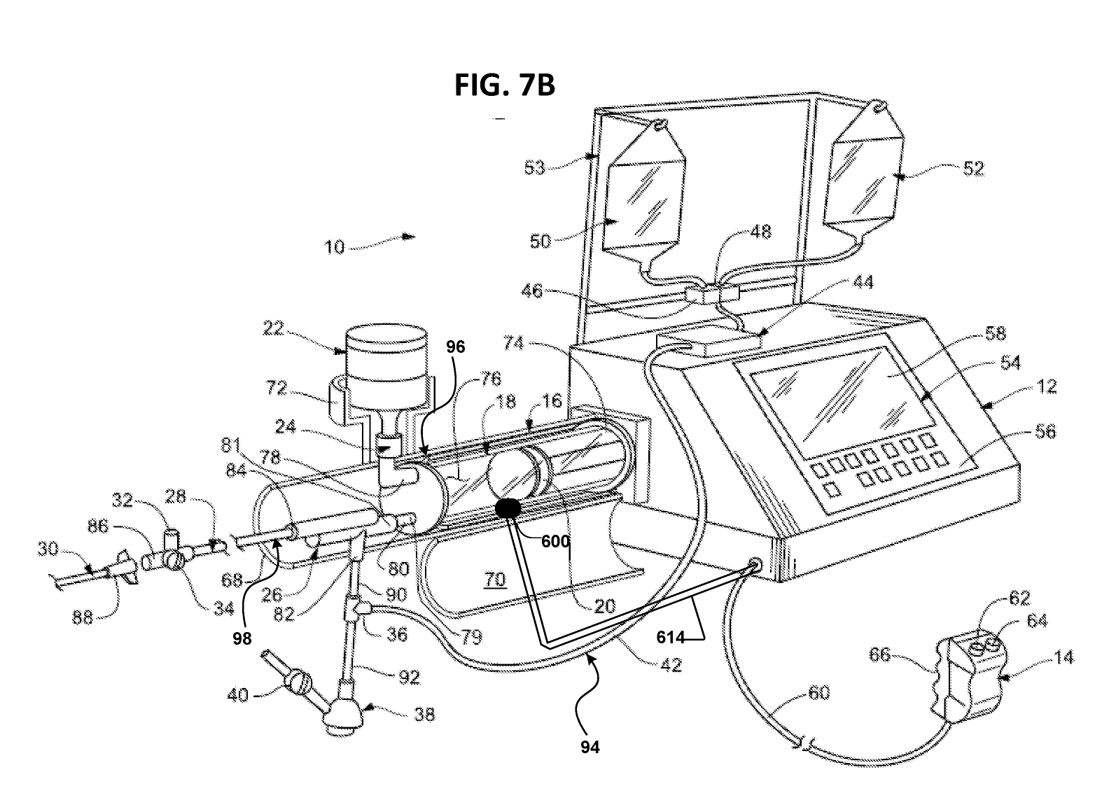

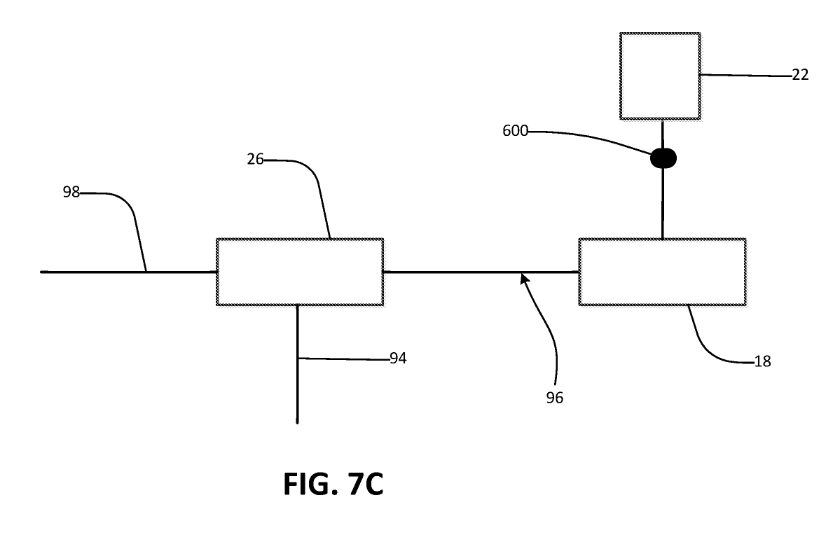

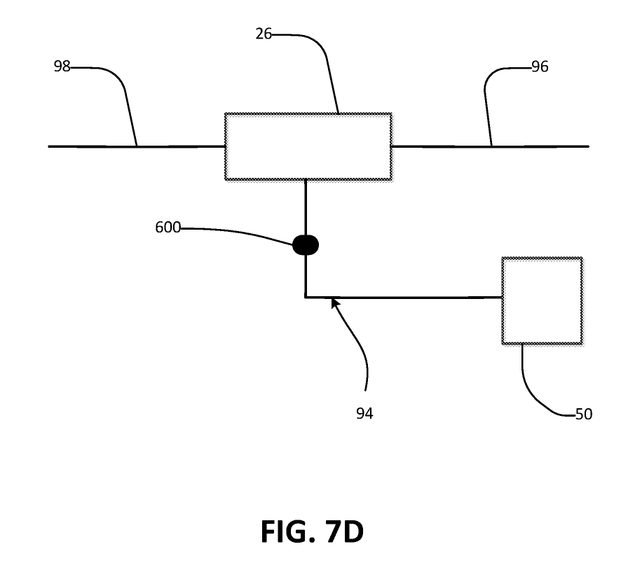

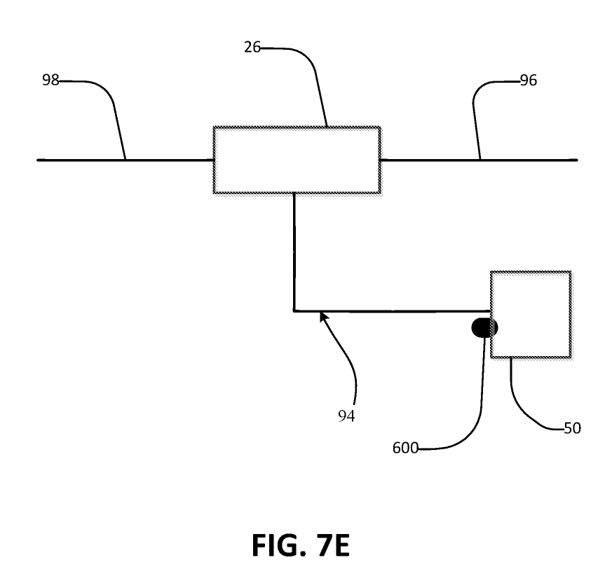

In addition to, or as an alternative to, use of a sterilization device and/or vibration device as described, still other devices can be included in embodiments of a contrast injector system to address potential cross-patient contamination. FIGS. 7A-7C illustrate use of an illuminator device in embodiments of a contrast injector system. The illuminator device can be useful, for instance, in detecting a contaminated fluid path.

FIG. 7A shows a schematic diagram of an embodiment of an illuminator device 600. The exemplary illuminator device 600 shown can include a housing 602 having a first housing surface 604. Within the housing 602 may be a light source 606 and/or a detection device 608. In some examples, the light source 606 can be any device capable of producing laser-induced fluorescence of one or more contaminants (e.g., blood contaminants, crystals). One exemplary light source 606 can include a laser configured to emit light energy within the blue band of the electromagnetic spectrum, such as at a wavelength between 410 nm and 400 nm, and more particularly in some cases at a wavelength of approximately 405 nm (e.g., a “blue” laser). The detection device 608 can be, for example, any device capable of detecting fluorescence caused by the light source 606, and can include, for instance, a photomultiplier tube adapted to detect fluorescence caused by a blue laser light source.

As shown in the illustrated example of FIG. 7A, the light source 606 and/or the detection device 608 can be located within the housing 602 so as to be proximate to a surface 610 of an interfacing component 612. In particular, where the first housing surface 604 interfaces (e.g., is in apposition) with the surface 610 of the interfacing component 612, the light source 606 and/or the detection device 608 can be disposed on (e.g., contact) the first housing surface 604 as shown in FIG. 7A. Such a configuration can facilitate effective fluorescence and subsequent detection of one or more contaminants within the interfacing component 612.

In operation of the illuminator device 600, the light source 606 and detection device 608 can work together to provide an indication of a contaminated interfacing component 612. The light source 606 can be turned on and act to cause one or more contaminants present within the interfacing component 612 to fluoresce. Fluorescence within the interfacing component 612 is sensed by the detection device 608. By configuring the light source 606 to cause contaminants within the interfacing component 612 to fluoresce and the detection device 608 to sense such resulting fluorescence, contamination within the interfacing component 612 can be identified and proper action to address this contamination can be taken (e.g., replacing the interfacing component 612 with a new component). This is particularly useful where a contrast injector system employs both single-use and multi-use components.

In some examples, the detection device 608 can include a processor for determining a size of a particle when fluorescence within the interfacing component 612 is sensed by the detection device 608. For instance, the detection device 608 can include a memory component in communication with the processor for storing one or more predetermined dimensional thresholds for particles sensed during activation of the light source 606. The processor can receive data sensed by the detection device 608 and determine from such data an approximate size (e.g., diameter) of a particle fluorescing within the interfacing component 612. The processor can receive the one or more predetermined dimensional thresholds for sensed particles from the memory and compare the determined size of the sensed particle(s) to the predetermined dimensional threshold(s). In some such examples, the data sensed by the detection device 608 during activation of the light source 606 can be communicated to the console where the described processor and memory can be located. In this embodiment, the console then processes the data and determines an approximate size of a particle fluorescing and compares the determined size of the sensed particle(s) to the predetermined dimensional threshold(s).

In either case, the illuminator device 600 or the console can output an indication based on the comparison between the determined size of the sensed particle(s) and the predetermined dimensional threshold(s). For instance, such indication can be output when the determined size of the sensed particle is equal to or greater than the predetermined dimensional thresholds. This output indication can serve to alert to potential contamination within the interfacing component 612, such as where a particle is present and of a size that generally should not be located within the interfacing component. In various examples, the predetermined dimensional threshold(s) for particles sensed during activation of the light source 606 can be within a range of 10 to 1000 microns. In one such example, where the predetermined dimensional threshold is 10 microns, the illuminator device 600 (or the console) can output an indication when the determined size of the sensed particle is equal to or greater than 10 microns. Use of the illuminator device 600 to determine a size of a particle when fluorescence within the interfacing component 612 is sensed can be useful in detecting the presence of crystals, blood, etc. within the interfacing component 612.

FIGS. 7B-7E are schematic diagrams illustrating embodiments having differing locations of the illuminator device 600. FIG. 7B shows the embodiment of the system 10 described previously, in which like numbers represent like features. In FIG. 7B the illuminator device 600 (shown schematically in FIG. 7B) is included in the system 10. In the present example, the illuminator device 600 is disposed on a surface of the reservoir main body 18 (e.g., contrast reservoir), such that the reservoir main body 18 is the interfacing component described in FIG. 7A and the light source of the illuminator device is positioned to illuminate the reservoir main body 18. Thus, the light source and detection device of the illuminator device 600 can be disposed proximate to (e.g. contact), via the housing surface, the surface of the reservoir main body 18 to induce fluorescence of one or more contaminants and subsequently detect such fluorescence within the reservoir main body 18.

As also shown in FIG. 7B, the illuminator device 600 is in communication with the console 12 via line 614. For instance, the illuminator device 600 can be in signal communication with the console 12 via the line 614 such that the illuminator device 600 is controllable by the console 12. In other embodiments, the illuminator device 600 can be in signal communication with the console 12 via wireless transmission means (e.g., a local area network). Signal communication between the illuminator device 600 and the console 12 can be two-way communication, allowing for the console 12 to send various commands to the illuminator device 600 as well as to receive various signals from the illuminator device 600. In some embodiments, the illuminator device 600 can receive power over the line 614 from the console 12, in addition to signals from the console 12. Thus, the illuminator device 600 can in some embodiments be integrated with the system 10.

Exemplary commands which the console 12 can be configured to communicate to the illuminator device 600 can include a start and/or stop illumination command, a change illuminator parameter command, and/or an illuminator status check command. The start and/or stop illumination command sent from the console 12 to the illuminator device 600 can act, for instance, to begin and/or end emission of light energy from the light source, such as turning the light source on and/or off. In some examples, the start and/or stop illumination command may also act to turn the detection device on and/or off. The change illuminator parameter command sent from the console 12 to the illuminator device 600 can act, for instance, to adjust a wavelength of light energy emitted by the light source and/or adjust a time duration for which the light source is to operate. The illuminator status check command sent from the console 12 to the illuminator device 600 can act, for instance, to solicit a return signal from the illuminator device 600 to the console 12 containing requested data. For example, the console 12 can send a signal to the illuminator device 600 requesting data pertaining to one or more components of the illuminator device 600 (e.g., power supply, light source, detection device), and the illuminator device 600 can send a return signal to the console 12 containing data pertaining to one or more components of the illuminator device 600 (e.g., status of power supply, status of light source, status of detection device). Thus, in some examples communication from the illuminator device 600 is in response to communication received from the console 12.

In further examples, communication between the illuminator device 600 and the console 12 can include detected fluorescence related data. For instance, the detection device, via the illuminator device 600, may be in signal communication with the console 12. The console 12 can receive detected fluorescence data pertaining to contaminants or other matter illuminated by the light source within the interfacing component (fluid supply container 22 in the example of FIG. 7B). Depending on the detected fluorescence data received at the console 12, the console 12 may be configured to output a replacement indication based on the received detected fluorescence data sensed by the detection device. For example, the console 12 may process (e.g., using a processor within the console 12) the detected fluorescence data received from the detection device of the illuminator device 600 so as to compare the detected fluorescence data to a predetermined threshold contamination level for the interfacing component (e.g., fluid supply container 22). For embodiments where a predetermined threshold contamination level for the interfacing component is used, the change illuminator parameter command may also serve to adjust the predetermined threshold contamination level based on a particular application. If the detected fluorescence data is equal to or greater than the predetermined threshold contamination level, the console 12 can generate and output the replacement indication so that a user of the contrast injector system is made aware of a potential need to replace the interfacing component with a new component. In some cases the replacement indication may be conveyed to the console 12 in response to a request from the console 12, while in other cases the system can be configured to send detected fluorescence data from the illuminator device 600 to the console 12 automatically at preset time intervals.

In other examples (not shown), the illuminator device 600 can be an in-line accessory to a contrast delivery system. For instance, the illuminator device 600 may not be in communication with the console 12, but rather may have an integrated user interface allowing a user to input commands pertaining to operation of the illuminator device 600 directly to the illuminator device 600. The integrated user interface on the illuminator device may also include means for outputting detected fluorescence data. In such examples where the illuminator device 600 is an in-line accessory, the illuminator device may also have a distinct power input separate from the console 12.

FIG. 7C schematically illustrates an alternative position of the illuminator device 600 on the second fluid communication line 96, such that the light source of the illuminator device 600 can be positioned to illuminate the second fluid communication line 96. Thus, in the embodiment shown in FIG. 7C the illuminator device 600 is positioned such that the interfacing component is now the second fluid communication line 96 (e.g., the light source and detection device within the housing of the illuminator device 600 are proximate to a surface of the second fluid communication line 96). As shown, the illuminator device 600 is positioned on the second fluid communication line 96 upstream of the reservoir main body 18, but in other examples the illuminator device 600 can be positioned on the second fluid communication line 96 within the reservoir main body 18 or downstream of the reservoir main body 18. In operation, the illuminator device 600 can utilize the light source and detection device within the housing to cause matter, such as contaminants, within the second fluid communication line 96 to fluoresce and be detected. In a further example, two illuminator devices 600 can be utilized, with one illuminator device 600 on the surface of the reservoir main body 18 and one illuminator device 600 on the second fluid communication line 96 upstream or downstream of the reservoir main body 18.

FIG. 7D schematically illustrates an alternative position of the illuminator device 600 on the first fluid communication line 94, such that the light source of the illuminator device 600 can be positioned to illuminate the first fluid communication line 94. Thus, in the embodiment shown in FIG. 7D the illuminator device 600 is positioned such that the interfacing component is now the first fluid communication line 94 (e.g., the light source and detection device within the housing of the illuminator device 600 are proximate to a surface of the first fluid communication line 94). In operation, the illuminator device 600 can utilize the light source and detection device within the housing to cause matter, such as contaminants, within the first fluid communication line 94 to fluoresce and be detected.

FIG. 7E schematically illustrates an alternative position of the illuminator device 600 on the fluid supply container 50 (e.g., saline supply container), such that the light source of the illuminator device 600 can be positioned to illuminate the fluid supply container 50. Thus, in the embodiment shown in FIG. 7E the illuminator device 600 is positioned such that the interfacing component is now the fluid supply container 50 (e.g., the light source and detection device within the housing of the illuminator device 600 are proximate to a surface of the fluid supply container 50). In operation, the illuminator device 600 can utilize the light source and detection device within the housing to cause matter, such as contaminants, within the fluid supply container 50 to fluoresce and be detected.

The illuminator device shown in FIGS. 7C-7E may be in communication with the console as described previously. In various examples, multiple illuminator devices 600 can be used in two or more of the described locations of FIGS. 7B-7E. Where more than one illuminator device 600 is utilized, the console 12 can be in independent and/or synchronized communication with each of the sterilization devices 600.

In some embodiments, one or more of the illuminator device 600, sterilization device, and/or vibration device can be used in a single contrast injector system, including more than one of a particular illuminator, sterilization, and/or vibration device. For example, a single contrast injector system can utilize a sterilization device and vibration device to facilitate both eradication and prevention, respectively, of contaminants as well as an illuminator device to facilitate detection of a contaminated fluid path. This may, for instance, allow the contrast injector system to operate more efficiently. For instance, the system could be programmed at the console to selectively power only certain of the utilized devices based on communications received from one or more of the devices.

Embodiments of the invention can further include methods related to use of a contrast injector system described herein. For instance, such methods can relate to a contrast injector system having a sterilization device, vibration device, and/or illuminator device as detailed herein.

Exemplary methods can include sterilization of a section of a fluid path within a contrast injector system. Such methods can include positioning a sterilization device to emit energy to a component of the contrast injector system, such as a fluid supply container, fluid communication line, and/or conduit. Positioning the sterilization device may include placing at least a portion of the fluid communication line and/or conduit within a housing channel of the sterilization device, such that the energy emitter directly interfaces with (e.g. contacts) the fluid communication line and/or conduit within the housing channel. In addition, these methods can involve communicating with the sterilization device from the console of the system to convey one or more commands, such as commands relating to emission of energy from the sterilization device.

Further method embodiments can include inducing vibrations on a surface of a component in a contrast injector system. Such method can include positioning a vibration device to induce vibrations on a surface of a component of a contrast injector system, such as by positioning the vibration device on a surface of a fluid supply container or on a fluid communication line or conduit. Positioning the vibration device may include positioning the vibration device such that a piezoelectric actuator within a housing of the vibration device is proximate to the surface on which vibrations are to be induced. In addition, various such methods can further include communicating with the vibration device from the console of the system to convey one or more commands, such as commands relating to actuation of the piezoelectric actuator and/or commands relating to operation parameters of the vibration device (e.g., operation frequency).

Additional method embodiments can include causing matter within a component in a contrast injector system to fluoresce and be detected. Such methods can include positioning an illuminator device such that a light source of the illuminator device to illuminate a fluid supply container, fluid communication line, and/or conduit of the contrast injector system. Positioning the illuminator device may include positioning a light source of the device proximate to such component to illuminate the contents of the component. Fluorescence of matter within the component can be detecting using a detecting device within the housing of the illuminator device. In some method embodiments, detected fluorescence data can be communicated from the illuminator device detecting device to the console, where such fluorescence data can be processed. In one example, the console may out an indicator to a user that a component of the system needs to be replaced based on the received fluorescence data.

Various examples of the invention have been described. Although the present invention has been described in considerable detail with reference to certain disclosed embodiments, the embodiments are presented for purposes of illustration and not limitation. Other embodiments incorporating the invention are possible. One skilled in the art will appreciate that various changes, adaptations, and modifications may be made without departing from the spirit of the invention and the scope of the appended claims.

1. A contrast injector system comprising:
a manifold including a first fluid inlet, a second fluid inlet, a fluid outlet, and a valve, wherein the valve is configured to switch between allowing fluid communication from the first fluid inlet to the fluid outlet and fluid communication from the second fluid inlet to the fluid outlet;
a first fluid communication line connecting a first fluid supply container in fluid communication with the first fluid inlet of the manifold;
a second fluid communication line connecting a second fluid supply container in fluid communication with the second fluid inlet of the manifold;
a reservoir main body positioned on the second fluid communication line and configured to receive fluid from the second fluid supply container and communicate the received fluid to the second fluid inlet of the manifold;
a console configured to control an operational parameter of the reservoir main body;
a conduit in fluid communication with both the fluid outlet and a vasculature of a patient; and
a sterilization device in communication with the console and having an energy emitter, and wherein the energy emitter is positioned to emit energy to i) the conduit, ii) the first fluid communication line, or iii) the second fluid communication line.

2. The system of claim 1, wherein the sterilization device includes a housing defining a housing inlet, a housing outlet, and a housing channel extending through the housing from the housing inlet to the housing outlet, the housing channel configured to receive i) the conduit, ii) the first fluid communication line, or iii) the second fluid communication line.

3. The system of claim 2, wherein the housing includes a first housing portion and a second housing portion, and wherein the housing is configured such that the second housing portion is movable to the first housing portion to provide access to the housing channel.

4. The system of claim 3, wherein the first housing portion and the second housing portion are hingedly connected.

5. The system of claim 1, wherein the sterilization device is in signal communication with the console such that the sterilization device is controllable by the console.

6. The system of claim 5, wherein the console is configured to communicate to the sterilization device a start/stop sterilization command, a change sterilization parameter command, and a sterilization status check command.

7. The system of claim 1, wherein the energy emitter of the sterilization device is configured to emit ultraviolet radiation.

8. The system of claim 7, wherein the ultraviolet radiation includes wavelengths between 280 nm and 100 nm.

9. The system of claim 1, wherein the reservoir main body is configured to pressurize the received fluid from the second fluid supply container such that fluid communicated through the second fluid inlet of the manifold is at a higher pressure than fluid communicated through the first fluid inlet of the manifold.

10. The system of claim 9, wherein the operational parameter of the reservoir main body that the console is configured to control is the pressurization of the received fluid from the second fluid supply container.

11. The system of claim 9, wherein the valve comprises a spool valve, wherein the spool valve is spring biased so that the first fluid inlet is normally connected to the fluid outlet, and wherein the reservoir main body is configured to pressurize the received fluid from the second fluid supply container to a degree sufficient to overcome bias force against the spool valve so that the second fluid inlet is connected to the fluid outlet.

12. The system of claim 9, wherein the second fluid supply container includes a supply of contrast media.

13. The system of claim 12, wherein the first fluid supply container includes a supply of saline.

14. The system of claim 13, wherein the energy emitter is in contact with the conduit.

15. The system of claim 13, further comprising a check valve positioned along the first fluid communication line and configured to prevent fluid communication from the first fluid inlet of the manifold into the first fluid supply container, wherein the energy emitter is in contact with the second fluid communication line.

16. The system of claim 13, wherein the console includes a motor for driving a plunger of the reservoir main body to pressurize the received fluid from the second fluid supply container.

17. A contrast injector system comprising:
a manifold including a first fluid inlet, a second fluid inlet, a fluid outlet, and a valve, wherein the valve is configured to switch between allowing fluid communication from the first fluid inlet to the fluid outlet and fluid communication from the second fluid inlet to the fluid outlet;
a first fluid communication line connecting a first fluid supply container in fluid communication with the first fluid inlet of the manifold;
a second fluid communication line connecting a second fluid supply container in fluid communication with the second fluid inlet of the manifold
a reservoir main body positioned on the second fluid communication line and configured to receive fluid from the second fluid supply container and communicate the received fluid to the second fluid inlet of the manifold;
a console configured to control an operational parameter of the reservoir main body;
a conduit in fluid communication with both the fluid outlet and a vasculature of a patient; and
a vibration device in communication with the console and disposed i) on a surface of the second fluid supply container, or ii) on the second fluid communication line.

18. The system of claim 17, wherein the vibration device is disposed on the second fluid communication line at a surface of an inlet port, and wherein the inlet port is a portion of the second fluid communication line extending between the second fluid supply container and the reservoir main body.

19. The system of claim 17, wherein the reservoir main body is configured to pressurize the received fluid from the second fluid supply container such that fluid communicated through the second fluid inlet of the manifold is at a higher pressure than fluid communicated through the first fluid inlet of the manifold.

20. The system of claim 19, wherein the second fluid supply container includes a supply of contrast media and the first fluid supply container includes a supply of saline.

21. The system of claim 17, wherein the vibration device includes a housing with a piezoelectric actuator disposed within the housing.

22. The system of claim 21, wherein the piezoelectric actuator is disposed within the housing of the vibration device proximate to the surface of the second fluid supply container or the second fluid communication line.

23. The system of claim 22, wherein the housing includes a surface in apposition to the surface of the second fluid supply container or the second fluid communication line, and wherein the piezoelectric actuator is disposed on the surface in apposition to the surface of the second fluid supply container or the second fluid communication line.

24. The system of claim 21, wherein the piezoelectric actuator is a low-frequency piezoelectric actuator configured to operate at a frequency between 100 kHz and 300 kHz.

25. The system of claim 17, wherein the vibration device is in signal communication with the console such that the vibration device is controllable by the console.

26. The system of claim 25, wherein the console is configured to communicate to the vibration device a start/stop vibration command, a change vibration parameter command, and a vibration status check command.

27. A contrast injector system comprising:
a manifold including a first fluid inlet, a second fluid inlet, a fluid outlet, and a valve, wherein the valve is configured to switch between allowing fluid communication from the first fluid inlet to the fluid outlet and fluid communication from the second fluid inlet to the fluid outlet;
a first fluid communication line connecting a first fluid supply container in fluid communication with the first fluid inlet of the manifold;
a second fluid communication line connecting a second fluid supply container in fluid communication with the second fluid inlet of the manifold;
a reservoir main body positioned on the second fluid communication line and configured to receive fluid from the second fluid supply container and communicate the received fluid to the second fluid inlet of the manifold;
a console configured to control an operational parameter of the reservoir main body;
a conduit in fluid communication with both the fluid outlet and a vasculature of a patient; and
an illuminator device in communication with the console and having a light source, and wherein the light source is positioned to illuminate i) the first fluid supply container, ii) the first fluid communication line, iii) the second fluid supply container, or iv) the second fluid communication line.

28. The system of claim 27, wherein the light source of the illuminator device includes a laser configured to emit light energy at a wavelength between 410 nm and 400 nm.

29. The system of claim 27, wherein the illuminator device further includes a detection device configured to detect fluorescence of matter illuminated by the light source within i) the first fluid supply container, ii) the first fluid communication line, iii) the second fluid supply container, or iv) the second fluid communication line.

30. The system of claim 29, wherein the illuminator device includes a housing with the light source and the detection device disposed within the housing, wherein the light source and the detection device are disposed within the housing of the illuminator device proximate to a surface of i) the first fluid supply container, ii) the first fluid communication line, iii) the second fluid supply container, or iv) the second fluid communication line.

31. The system of claim 29, wherein the detection device is a photomultiplier tube.

32. The system of claim 29, wherein the detection device is in signal communication with the console such that the console receives detected fluorescence data pertaining to matter illuminated by the light source.

33. The system of claim 32, wherein the console is configured to output a replacement indication based on the received detected fluorescence data pertaining to matter illuminated by the light source.

34. The system of claim 32, wherein the console is configured to communicate to the illuminator device a start/stop illumination command, a change illuminator parameter command, and an illuminator status check command.
